(19) United States
(12) Patent Application Publication
(10) Pub. No.: US 2023/0172682 A1
(43) Pub. Date: Jun. 8, 2023

(54) Discrete Cryosurgical Primary-Device

(71) Applicant: Rory Farlan Krieger, Lakewood, CO (US)

(72) Inventor: Rory Farlan Krieger, Lakewood, CO (US)

(73) Assignee: ABC-PRESENTS, LLC., Lakewood, CO (US)

(21) Appl. No.: 17/643,135

(22) Filed: Dec. 7, 2021

(51) Int. Cl. A61B 90/00; A61B 18/02

(52) U.S. Cl. CPC A61B 90/04; A61B 18/0218; A61B 2018/0047

(57) ABSTRACT

Mechanical biomimicry translator. Epidermal lesion-removal triage-platform. Present invention cryotreats humanity suffering from benign basal-cell to aggressive, malignant, oncological lesions. Hand-held, artisan-styled monoframe. Prosthetic Blitzkrieg processing. Dermatology-tool.

Deployment eviscerates common histological abnormalities. Holographic Skeletal-Reflex Exoframe. Clinical. Metered. Structured. 360° rotate/ablate. Backbone. Glide-rails. Asymmetrical, gain-of-function; filtration-membrane.

Ground-zero; mobile directed-energy-matrix. Level-2 Spec-Ops-7. Digital encoder modulates/dispenses 3^{rd} party biologics. Multi-factor authentication; oncological, epidemiological, gynecological; Gender-balanced. L.G.B.T.Q.I.A+ compatible. Agnostic. Mercy-Aid. NGO.

Rapid-Defense, Rapid-Offense-Procedures. De-risking cryodestruction. Multi-mission capable; first-aid, mobile family-medicine; medical-tourism. Pocket-Sherpa form-factor. Panoramic lesion harvesting. Recyclable.

Essential acute-care; lowering patientcare costs. Inclusion, diversity, equity, action. Uncompromising mitigation strategy. Comprehensive menu, human-performance enhancements democratizing open-source, Krieger-Amulet-TaaS®. Plug and play cold-fusion cryosurgery. Interdiction-Consumer-Empowerment. Humanitarians without borders. Patient-Lives-Matter.

### STATEMENT REGARDING FEDERALLY SPONSORED RESEARCH OR DEVELOPMENT N/A REFERENCE TO SEQUENCE LISTING

N/A

### TECHNICAL FIELD OF INVENTION

The present invention relates generally to medical devices to accurately cull, and ablate unwanted biologic tissue(s) at targeted sites. Specifically, relating to cryotreatment devices and methods for inducing controlled necrosis of: moles, lesions, flat-warts, morphologies etc.; imparting a measured-ration of a cryogen. Responsible, safe, personalized-patientcare; treating humanity suffering from harmless basal-cell, to life-threatening cancer. Skills-based paradigm-shift; a Next-Gen, B2B/B2C medical asset for stakeholders who perform quality, out-patient triage.

### BACKGROUND DISCLOSURE OF INVENTION

Rory Krieger is the sole named inventor for a medical device known as: Discrete Cryosurgical Primary-Device™ (DCP-D). “A fitting turn-of-phrase to describe the medical merits of a top-tier, a la carte medical device. Indicative of an invention to which a menu of Claims is targeted for cryosurgery.”/RK

How did we get here? For said inventor, the origins of this device dates back to his formative teen-age years. “A long-suffering patient at several area dermatology clinics and family MD's where my skin-tags were under-treated & recklessly over-sprayed. Each doctor had a poor aim. Unable to shoot-straight with on-hand clinic tools. Inflicting scars. PTSD fears! MD's follow-up offering dismissive vacuous excuses, before handing me an up-coded bill! Time-after-time misguided practitioners pushed their thumbs against-the-scales;” opines inventor, Rory Krieger. “Inside Dr. Doolittle's ‘kabuki theater’ I winced. One skip-shot botched-triage (after another) from squads of lab-coat tyrants. Brazenly overplaying their hand. Naked aggression; misguided results. Out-patient triage became a PTSD white-knuckle ride. As a whistle-blower, several MD's & NPP's were flagged; siting contempt, malpractice by dystopian SOP's. Myopic. Detached from reality. Exposing dark-secrets in a clinical cottage-industry. Routinely failing naïve patients and up-coding their CPT bill.”

“Clinic-tools should be protective, compassionate, safe and inclusive. Reporting for duty, patient-centric, doing no harm. Replete with defined, well-documented CAPA (Corrective and Preventative Action) 820.100(a) (3). Legitimate safeguards with predictable outcomes. I discovered just the opposite! My dermatologists facilitated bait & switch, as a pretext for up-coded billing: half-measures, an absence of higher-quality-assets. An inconvenient truth emerged. They failed to resolve my own annual predilection of moles and warts. I still remember walking away murmuring, “They're running a parlor-game in there. I'm paying for collateral damage, brazen-ineptitude from defiant, dispassionate, smug autocrats. ADHD and tone-deaf. Quacks on-the-dole! A kitchen-table bread and butter issue for millions. Why on earth do we keep subsidizing our own demise? I learned a lot from that abuse. Patients have died. Their families now mourn. Their lives will never be the same. But I persevered. Bootstrapped. Skin-cancer became ‘the battlefield’. Sounding the alarm, I became a Patient-Rights-Activist for a pragmatic, better-safer-faster patientcare experience. A ‘Blitzkrieg Good-will Ambassador’; liberating an oppressed patronage. Advocating a quality Med Tech device to serve professionals and DIY stakeholders on the front-lines. Prevent more pain and suffering for patients who just want a fair shake.

The epithelial/epidermal layer of skin. Raising the bar for: Inclusion, Diversity, Equity, Action (IDEA). “/R.Krieger Some view healthcare providers as saviors; others as conscripts, armchair providers wolves in sheep's clothing. Long-overdue, one newcomer addresses safety and security assistance. As for this botched triage survivor (USCG/USAF serviceman), “some healthcare professionals wield authority and clinical-tools not used in the manner intended”. Leading from behind, unbecoming of their sworn profession. Violating the spirit of “do no harm.” Stymied, many employ unsafe, scorched-earth, reckless-pressurized CO2 sprayers where toxic Freon gasses blast through a tube, dispersed into a cone speculum; positioned around abnormal tissue (pooling the coolant). In other methods, a supply-tube uses a porous-tip-applicator. More commonly, a cotton Q-tip soaked-in LN2 is directly applied to the surface of the lesion (a safer measure).

Sadly, pressurized sprayers are complex, fragile accident-prone. Nozzles stick-open! Injurious to the patient. Embedded within the cottage-industry, physicians and prior art devices have a cozy, quid pro quo supplier/user/payer relationship. Patient bills routinely up-coded, inflated outside the industry “CPT Integumentary Coded System” (CPT). Technologies providers commonly use: comically opaque. Inviting user error: blemish streaks. Disturbing macabre; red-burn blisters, numerous return visits to fix botched triage. It's a perfect-storm where patient injury+casual malpractice is common. A systemic top-down problem. A provocation that under-scores the human-toll in this cottage industry. A “ground-war” heavily reliant on guess-work.

Antiquated SOP's+deficient medical tools=botched results. A chilling-reminder how conscripts, NPPs and MDs willingly spray lethal-doses of chemicals at patients, without properly masking.

Failing to isolate (mask) lesions from adjoining virgin tissue! Naked aggression, neglecting patient's Informed Consent. Worth noting, IVD-injections, chemo-peels, cauterizing and biopsies is commonly over-performed as well. At the end of the day, a minority of healthcare proxies walk-away. Leaving victimized, inhumane, keloid-scarred patients in their wake! over and over again, patient's skin-blistered, scarred. Sore and stigmatized. Socially embarrassed from a simple out-patient triage, gone-bad.

As for this inventor? His reward was a pat across the back, and a CPT up-coded bill! Another patient caught in the cross-fire; another patient bit the dust. Spotlighting a troubling-pattern of hubris, flagrant abuse. Generations in the making, this is the predicament patients are now finding themselves in. The stuff of clinical-nightmares. Critical mass. An example of life imitating art. Marking this, his own Ground-Zero moment.

A cold-war marked by mercurial moments. Posturing. Situation was tense. Internal warning bells led Krieger into “invent-mode” and social-media. According to sources, legions of long-suffering patients are writing open-letters. Embattled over nixed-triage from local dermatology clinics. Many cited by consumer-watch groups for blatant safety violations. A slippery-slope where patients are pumping their fists with raw emotions; some dying. Many openly confess to avoiding annual doctor exams citing their own PTSD fears, botched triage, visible L/T surface scarring. Chalk it up to unapologetic doctor's privilege, professional-immunity, up-coded billing, provider half-measures. A top-down, ill-equipped grip on outdated practices; led to one bottom-up, grass-roots fix. A skeletal, flexible slide-polymer mono sled-frame. A mission capable, “semi-automatic, LN2-socket Med Tech tool. “Discrete Cryosurgical Primary-Device” (DCP-D/ADV-01). DCP-D is a common-sense, all-in-one, patientcare triage system.

Following decades of repeated abuse, the time came for Krieger to lend his voice. Emboldened with fearless determination. Speaking truth-to-power on behalf of all abused patients. Breaking old-rules is how activism . . . will right-the-ship. Democratizing healthcare under a cache of Intelligent Novel Assets-On-Demand (CACHE-IN-AOD). A device with Corrective and Preventive Action (CAPA); ISO and FDA regulations built-in. Triage by the numbers, a “depot of novel safety features” providing a “mechanized forward deployed tool; supporting a masked-mandate, and ICE.” A Med Tech SecureFIT solution. A Level-2 technology having AI/AR efficiency, promoting aperture-suppressor shielding, saving virgin-tissue. Supporting authentic tenants of dermatology, and the tenor of FDA regulatory standards. DCP-D aspires to be a strategic carve-out tool in an industry desperate “to stop the bleeding and patient desertion.” Using mechanical sockets and naturally occurring “organic biomimicing shapes” to get the job done. A Technology as a Service (TaaS), plug-and-play model. Mending what is broken within ADA/AMA cryodestruction triage. A safety improvement over prior-art, predicate and 510(k) devices.

DCP-D supports: Quality Management Systems (eQMS), Regulatory Pathway Assessment (RPA), Design for Manufacturing (DFM); Human Factor Design Controls (HFD-C). Clinical R&D. Stakeholder-centric checks and balances, and FDA audits.

DCP-D is a tactical, touch and go, Rapid-Triage-Device (RTD); homing-in with gain-of-function precision and CRT friendly-fire accuracy. Ending tone-deaf healthcare abuses by: a. dangerous and deficient prior art devices, b. archaic/lagging SOP's.

A practical panacea for teens, millennials and seniors who face an uncertain future, yet need elective surgeries during COVID.

Reporting for duty, DCP-D provides leadership to stakeholders. DCP-D uses equity-based Med Tech ingenuity for clean, trouble-free, post-op ADA/AMA results.

Overcoming prior art, predicate and 510(k) deficiencies. A forward strategic-platform for novice, to board-certified MD providers. DCP-D (ADV-01) is a tactical, asymmetric-approach under modern FDA, QSIT Guided standards. A technician's-tool specializing in 1^{st} responder patientcare, engineered to safely remove basal-tissue, and aggressive skin-cancer lesions (on epithelial/epidermal surfaces). A “Level-2, GT Unlimited-Class” hand-held asset, DCP-D promotes a “one-shot treatment without circling-back for repeated triage.” Controlling the much abused CPT up-coding (subterfuge billing). Personalized healthcare in a safe and orderly-manner. Extending elective surgery opportunities for Humanitarians without Borders; relief from economic disruptions due to a pandemic.

Cryosurgery is the destruction of tissues using biologic combination means; directed at lesions and morphologies on the epidermis (epithelial surface layers) of living skin (mucous membranes); targeting blood vessels supplying the lesion. Leveraging the use of LN2 @ −319° F., producing snow-ball ice crystals inside outgrowth cells; resulting in 100% necrosis. Lesion temperatures are dependent upon: phenotype, delivery means and method by differing biologic cryogens; plus skin temperature prior to treatment, OR-room temperatures; patient histology. Many individual and specialized factors are at play including HIPPA laws, Informed Consent, racial inequities, etc.

Removal by “destruction technique” is ADA defined as . . . “the removal of benign, pre-malignant, integumentary system tissue. Removal by any method (electro surgery, cryosurgery, laser and chemical treatment). With or without curettement; local anesthesia that does not require mechanical closure.” A procedure similar to a Holographic Augmented Reality (HAR) otoscope; specula cryosurgery; using DCP-D's bespoke Level-2 AI/AR nomenclature, under the rubric of a “lesion masked-mandate as a border-wall” that seals-in suspect/unwanted lesions, from native, homologous virgin-tissue via Human-Factor engineering.

Triage services are reported to Medicare/Medicaid, using CPT coded groups [2013 AMA CPT® Codebook: 1700x, 1711x; and 1726x]. Updated by International Statistical Classification of Diseases and Related Health Problems (ICD); World Health Organization.

Technical Problem: Dermatology Devices Hit a Brick Wall

In a face-to-fact comparison with DCP-D, we quickly learn how prior art attempts are non sequiturs. Aside from the eQMS and inputs/outputs/indications realm, many prior art predicates employ remedial, Draconian methods. A wild shot-gun approach. Missing the target. Butchering patient skin with chemicals, maligned SOPs. Lacking CAPA and ethical standards of care. Limited, irresponsible, failing traceability and discrete mannerisms. Current SOP's actually allow for botched treatment; prioritizing shady remittances over personalized patient-care. Another failing comes in their skip-shot shotgun-blast spraying method; use of highly-pressurized aerosols, and fragile-hair-trigger materials. Leading to a short life-cycle. And there's this, toxic permeability and leaching. Toxic GMO transfer. Clinical ambiguities. Infections. Heightened patient-anxiety. Malpractice fear mongering. Missing-out on Elective Procedures. Putting-off annual health screening/exams. Placing patients in the cross-hairs; facing a bleak and uncertain diagnosis.

SOPs+Systemic Tooling Failures Plague Cryosurgery Industry Botched triage, systemic injury and malpractice is directly correlated to those industry-wide failings, and the “hang-over effect” where patients cannot gain access to quality medical treatments. Due in part to the SARS-CoV-2 strain of coronavirus.

Reports reveal prior art challengers develop uncivilized, accident-prone devices with porous-borders, creating erroneous, often injurious results. Failing to bind-up a nation in pain. Mulligans plague this cottage-industry. Evil flourishes, when good men do nothing. A frontier, a cottage-industry climate where one Med Tech tool, aspires to democratize an industry. Bringing to an end the blood-letting. Malpractice. Pain-suffering. Treating morphologies responsibly.

According to an April 2018 Medicare Quarterly Provider Compliance Newsletter, “CMS's CERT contractors are reporting that they have seen an increase in E/M claims issued due to improper coding, insufficient notes, and setting confusion. In fact, ‘the 2027 improper payment rate for HIM services was 12.1 percent, accounting for 10.6 percent of the overall Medicare FFS (Fee-for-Service) improper payment rate’.”

As mentioned, improper cryotherapy is dangerous. Many botched treatments (including cauterization) leaves behind harsh, discolored, raised Keloid scarring (and PTSD trauma).

With doctors and patients now locked-in a cold-war of their own, DCP-D breaks-the-ice, without leaving critical medical surgery to chance. Breaking-ranks with SOPs and hazardous clinic tools.

Dermatology clinics store a staple of items in their coffers;

1. Liquid Nitrogen (LN2) gas in a Dewar or stainless thermos

2. A fresh bag of long, Q-tip cotton sticks

3. Needles and numbing Novocain for pre-treating affected areas

4. An electric cauterizing-rod for burning-off lesions

5. Autoclaving, UVA/UVB light, alcohol wipes, and/or a myriad of surgical, hospital and/or advanced sanitizing methods.

Over the decades, inventor-Krieger was a patient with a front-row, ring-side seat in “Doctor Doolittle's clinic.” Rushed, impetuous triage on-the-fly, became a dog & pony minstrel show. Infuriated and no longer a trusting babe in the woods, Krieger bravely under-took this challenge. Spawning R&D of the DCP-D. An FDA/ISO/ADA/AMA/CE/HIPPA compliant, Med Tech work-around.

MACRA stands for Medicare Access and CHIP Reauthorization Act. A Medicare payment law intended to govern health care payment and delivery system reform for clinicians, health systems, Medicare, and other government and commercial payers.

The law establishes a path toward a new Medicare payment system that will more closely align payment with quality of patientcare and outcomes. MACRA offers financial incentives for health care professional to participate in shared risk-bearing and coordinate varying healthcare intervention models; moving away from the antiquated, outgoing Fee-for-service (or FFS system). Physicians and other clinicians paid under the Medicare fee schedule will generally choose between participating in Alternative Payment Models (APMs); payment based upon individual performance; Merit-Based Incentive Payment System (MIPS).

### PRIOR ART (PREDICATE DEVICES BY CHALLENGER INVENTORS)

Regrettably, prior art predicates fail Stages 1 & 2; “one-dimensional weathervanes who devolved into opaque copy-cats.” Providing “no substantial, disruptive or next-level technologies.” No CAPA, no Augmented Reality (AR), no preventative Artificial Intelligence (AI) nor around-the-bend counter-measures to eliminate the dreaded skip-shot triage morass. It also bears mention that: topical salicylic & brush-on chemo-peels, homeopathic salves/acids and chemical nerve agents have proven problematic, causing pain and suffering to patients.

### Prior Art: A Pathology in Cryogenic Therapeutic Treatments

a. Challenger inventors in: application No. AU 2011101126 A4; Jared S. McCann, Melissa S. McCann describe how their Cryoshield device addresses the issue. McCann & McCann use a disposable plastic otoscope specula whereby the physician holds and presses the otoscope specula against a lesion; surrounding it for a Hail-Mary blast of liquid-death. Adding “ . . . The cone shaped specula are often trimmed to form a diameter suitable to surround the lesion.” The Cryoshield device is “a broken dam” stumbling under many on-the-fly design flaws. It completely lacks Rx discretion. “Trimmed to form a diameter” . . . involves cutting specula. Creating jagged-spurs. Incursions. Unwanted flesh-chopping edging. Vandalizing the specula is time-consuming and ill-advised (secondary process). Rendering it “damaged-goods, dangerous and unfit for reuse.” Lacking a credible, masked-mandate. Unable to properly shield/isolate virgin tissue.
   Trimmed, serrated-edging is injurious. Causing unspeakable, ripping-tearing-bleeding. Infections. Bad episodic triage: unforced-errors, collateral damage, pain and suffering.
   Indicative of the brutality in prior art designs. With no ballast, backbone-SRS or rapid triage assets in place; Piranha-like jagged-edges spells bad-blood for patients!
   Flagged “a rookie-tchotchke-gizmo” by the triage-weary general public; lacking the superior self-indexing, “Spec-Ops-7” IMP-key sockets (aperture compensators) of DCP-D. Cryoshield is a large, clunky, awkward, grotesque misfit. An unlikely doctor's office tool because it requires two-sets of hands to operate. Compounding matters, sharp edgy assembly uses thin, fragile constituent parts. Impossible to glide, body-surf, Switch-360° maneuver or probe between: legs, a sensitive groin, under breasts, skin-folds, behind the ear. Worse yet, risking eyelid canthus tags around the eye socket, nose or naval. Unable to bridge the gap, this device is suspect; resembling a crude, mid-evil weapon of punishment for the gun-shy, botched-triage-weary, local personage. Cryoshield is among many similar devices that have weaponized biological agents. A shock-and-awe outcome; under a “patient-care” pretext. Failing the patient-trust. Cryoshield lacks the rapid triage added-value mannerisms of DCP-D. Absent are: IMP portal-keys, CAPA/STEAM acumen; structured rules of engagement, and aperture barricades. In short, some may conclude this device has bitten the dust as an incredulous, to be avoided “medical device.”
b. Challenger inventors in: U.S. Pat. No. 6,226,996; Paul J. Weber, Robert S. Bader, Luiz B. Da Silva describe a Device for controlled cooling of a surface. “A device is described that uniformly cools a surface to a specified temperature using a mist of cryogenic fluid. The device comprises a cryogenic fluid reservoir, a valve for controllably releasing the fluid through an atomizing nozzle, a non-contact temperature sensor, and a control unit to display the measured temperature and optionally pre-set the desired surface temperature and control the valve. The application of mist can be controlled manually or electronically. This invention “provides a portable tool for safely cooling skin for dermatological applications.” Yet challenger's method is erratic, prone to failure, racked and ruined; whereby said device cannot focus nor narrow the danger-zone of its horrific, blinding death-spray assaults. Dressed to kill, this is a glorified aerosol dispenser that may easily be misused and misapplied. Damaging unaffected tissue without hesitancy, remorse; mea culpa or CAPA accountability. Limited by design inefficiencies. Having open-boarders; lacking ADA, CAPA-safeguard police.
c. Challenger inventors in: WO/2007/028975A1, Inventor Mohammed Firoz Hussein describes Improvements in or relating to cryogenic therapeutic treatments; an applicator for the dispensing of cryogenic fluid comprising a cup shaped element and exhaust valve; in use, the cup is held against the skin over the area to be treated. Cryogenic material is supplied via a tube from a cryogenic source such as an aerosol equipped with a valve “to effect a controlled release” of cryogen means. When the liquid gas is released, it collects within the ‘cup’ piece.
   Challenger's method is benign, generic. Another off-the-shelf death-sprayer. Where said device is nothing more than an attachment for a “final spray method.” Many hardships abound requiring the user to change cup sizes depending on the size of lesion to be treated. As seen before, this prior art method does not discriminate, nor meets the novel and unobvious rapid triage criteria assets of DCP-D.
d. Challenger inventors in: EP3669804A1; Inventor Bart Rossel describes “Actuator assembly for cryogenic Devices.” “The invention pertains to the technical field of cryogenic therapeutic devices, which devices facilitate the application of cryogenic therapeutic treatments. In particular, the present invention provides an actuator assembly for cryogenic devices, for easier and cleaner application of a cryogenic fluid.” A cryogenic applicator pen comprising an actuator assembly for controlling an outflow of a cryogenic fluid. Wherein said actuator assembly comprises at least one spring leaf (303), wherein said spring leaf (303) on one side bears against a ball (304) of a ball valve and on an opposite side bears against a positioning casing (302) bearing a needle (307) for piercing a cryogenic fluid cartridge. Device uses a needle which qualifies an IVD. Use of spring leaf and assembly further complicates device with a fragile assembly, having individual constituent parts. Challenger's method is simply a CO2 cartridge, indiscriminately spraying a barrage of liquid-death at patients “in their blast zone.” Supported by maligned, often-rushed, provider priorities. Failing the CAPA safety-measures, and discrete methodology of DCP-D. Inventor Bart Rossel has not blazed a new trail, nor adequately contributes to the intricate, discrete 3D-world of today's personalized performance medicine. Inventor's device is unimpressive. It fails to provide detailed, granular, or circumferential shielding around a lesion during dispensing a cryogas means. Failing the Cache of Intelligent Novel Assets-on-Demand (CACHE-IN-AOD). Including the multi-mission-capable, family medicine and/or MASH battlefield first-aid accoutrements of the DCP-D.
e. Challenger inventors in: U.S. Pat. No. 6,527,765 B2 Inventors Charles Kelman and Roman Perez-Soler, describe “Cryogenic Surgical System and Method of Use in Removal of Tissue;” developed a method of use for treating and removing unwanted tissue, preferably from an organ or other internal part of a patient. Adding that “in one embodiment, an ultrasonic device is used to ultrasonically vibrate the cryogenically-treated tissue to break apart the frozen tissue and the frozen broken-up pieces of tissue are removed using a technique, such as an aspiration process.”
   Challenger's method is an “IVD device.” Inventor creates a multiplicity of inherent design flaws (eQMS, CAPA, MIPS, violations); further complicated by an aspiration process. Failing Risk Reduction Techniques (RRTs) in cryotherapy. Serving an FDA IVD, Class-2 regulation. In the process, red-flagged for using actual patients as “human-shields.” Prior art is a text-book example of nascent pressurized devices doing a circus-like high-wire act, cloaked as internal surgery. All the while, creating a danger-zone that is harmful to naïve trusting patients who just want a fair shake; holistic healthcare. ISO of valid medical truth; purposeful transparency. Treatment that promotes equity with quality results. Treatment by a device that leaves nothing to chance. Placing all cards on-the-table.
f. Challenger inventors in: U.S. Pat. No. 9,492,216 B2; Inventors Gerals Fischer, Florian Hintringer, Martin Goll, describes their “Handle for an Ablation Device.”
   “Control handle adapted to be coupled to a positioning catheter, adapted to position in an object, and ablation control handle part adapted to be coupled to an ablation catheter.” Challenger's method is another IVD device. As such, fails to improve upon the epidermal lesion/morphology issue of basal-cell and pre-cancerous tissue growing on the epithelial, visible surface of the skin. Wherein, as an IVD, in-vitro probing treatment creates a substantial risk to a patient's life and limb; without the discretion attributes to treat common Nevis, flat-warts, peduncle hanging “finger” tags or foot-born Plantar fasciitis. Prior art is limited by open-borders. Remiss the Spec-Ops-7 IMP's of DCP-D. Lacking the cross-function componentry, 3D AI/AR holographic finesse; absent the heterogeneous bucchero; asymmetrical IMP-key mosaics. Absent the deep-reserves of prepositioned CACHE-IN-AOD assets, which DCP-D possesses.
g. Challenger inventor in: Cryoplate. When used with assignee Brymill; each “frusto-conical aperture” delivers a one-size fits all blast. Clumsy, lacking detailed granular ablation for smaller, medium, or large sized lesions. Especially vulnerable is ablation around: eye sockets, genitals, etc. A monstrosity. The size of a child's dinner-plate; it more closely resembles a shop-tool in a diesel mechanic's box. Inventor uses a thick, heavy Lexan plate. Featuring four conical holes: 4 mm, 7 mm, 9 mm and 12 mm. Awkward and heavy. Each funnel is compromised. Causing leakage, injury. Foul. Cryoplate is 20 mm tall; leakage and patient-injury prone. Gigantic at 4.75″ diameter. Clumsy, misshapen; industrial. Slippery, hard to hold. Out of sync to maneuver or navigate in or across: skin-folds, behind the ear, over eyelids, toes, genitals. Holes so big, you can drink out of them! Cryoplate is crude. Rife for collateral damage to nearby virgin tissue. Lexan is not autoclavable, marking this gizmo a Single Use Device (SUD). A non-green disposable. “Cold sterilization only” is inscribed on the device itself! Cryoplate lacks all common-sense protocols.

Crossing-the-line, Brymill Cryoplate® is dispassionate; failing all fidelity litmus-tests held dear by the DCP-D. Having no “in-between”nodules” or Level-2 MIT band either. Grotesque, the device is far too large to adequately fit or service a patient from the most sensitive, to open-areas. An offensive hack-job, creating more blunders than it can solve. It fails: AI/AR, rapid triage; the Cache of Intelligent Novel Assets-On-Demand (CACHE-IN-AOD) and a masked-mandate (of smart-amenities) built into the DCP-D. Cryoplatet is poorly designed. Its gaping funnels lack the “sealing undercuts” of the DCP-D. Marking Cryoplate® as mechanically unable, and incompetent at sealing-in the devastating biologic, Liquid Nitrogen (LN2). Malpractice in the making. Speculum apertures/nodules cannot: meter, govern nor control Liquid Nitrogen. Its limitations are numerous, prone to overload and over-pooling LN2. Failing numerous objective metrics which molest, scar and deface patients. Brazen, unapologetic and apoplectic, Cryoplate® serves as a vapid, uninspiring, wrecking-ball. Rounded speculum apertures are tone-deaf; perilous as open-borders. Destroying virgin skin. A wily-old campaigner, Cryoplate® is a “misfit-toy” in today's performance-based healthcare. A Public Health Hazard (PHH) that abridges the public trust while prone to medical malpractice litigation. In a manner of speaking “another one bites the dust!” Failing to match DCP-D's deep-bench. Deficient and inappropriate for triage. Lacking in: 3D HD, AI/AR holographic, CAPA cryotherapy. Comparatively, DCP-D uses a multitude of aperture sockets. Spec-Ops-7 alone measure: 2 mm, 3 mm, 4 mm, 5 mm, 6 mm, 7 mm and 8 mm! With many larger, tactical IMPs to spare; “sweeping-clean” 90%+ of unwanted-lesions, commonly found growing across normal patient bodies. Cryoplate® is Nolo contendere. Thus, DCP-D overcomes this device. Empowered by a plethora of: supportive, bilateral biomimicry. Using novel, Level-2 GT-Class assets. Discrete, ethical; safety-minded medical tooling. Producing customized patient-care for humanity.
h. Challenger inventor in: 3,664,344 Tyned Cryosurgical Probe; Michael D. Bryne describes, “Freezing of large tissue masses to significant depths is achieved by utilizing a tyned cryosurgical probe having sharpened tynes. Tynes that are embedded directly into the tissue to be frozen. Nitrogen is delivered in substantially liquid form directly to the exposed ends . . . ” In this example, Inventor describes an IVD device that punctures skin, inserting itself into a Class-2 distinction. A method that is limited to Class-2, and is prone to contamination and infection. Leading to untimely, preventable injuries. Inventor's probe is clumsy. Has multi-assemblies. Unable to provide 3D AI/AR assets for cryotherapy. Failing public safety imperatives.
i. Challenger inventor in: U.S. Pat. No. 9,974,684 B2 Method and Apparatus for Crogenic Treatment of Skin Tissue; Inventors Anderson, Manstein, Chan and Ting describe, “Cosmetic method and apparatus are provided that can provide cooling and/or freezing of skin tissue proximal to the skin surface to generate an appearance of reduced skin pigmentation. Skin can be cooled to a temperature less than approximately −5 degrees Celsius for a duration of about one minute or less . . . ” In this example, both inventors describe the premise of pigmentation lightening; not lesion removal. Thus, limitations are many. Chief among those, failing to remove unwanted cancerous tissue from patients,
j. Challenger inventor in: U.S. Pat. No. 9,750,556 B2 Cryothreatment Devices and Methods of Forming Conduction Blocks; Inventor Daniel M. Lafontaine describes, “Cryotreatment devices and methods of ablating tissue within the body are disclosed. A cryotreatment device in the cardiac muscle of the heart.” Limited by failing to improve common lesion removal.
k. Challenger inventor in: EP 2337 515 B1 Cryosurgical Device with Metered Dose; “The present invention relates generally to the field of cryosurgical devices for cryosurgically treating skin diseases and more particularly to a cryosurgical device for applying a cryogenic refrigerant from a pressurized container in metered doses.” Inventor is using a system for cryosurgically treating skin diseases and a method of using it on abnormal tissue. Inventor provides the “shot-gun . . . clear-the-zone” brutality approach, nothing more. Thus inventor's device is limited to boiler-plate, pressurized spray methods. Failing to improve upon copy-cat kindred processes. Unable to upgrade to: 3D AI/AR Level-2 cryogenics; or advanced skill-based triage on epithelial tissue in a safe, CAPA manner.

Murphy's Law+Prior Art=Doomed Confirmation-Bias Results

Dispassionate, emboldened. Uninspired and lacking humility; healthcare proxies have done their own bottom-line bidding. Prior art pundits inflate the value of their assets. Most employ harmful, misdirected widgets with constituent parts. Most have many attachment assemblies with fragile-bits. Complex multi-piece assemblies. Fail-prone, aerosol Trojan-horses embedded in many armchair clinics. Gas-lighting procedural gaffs. Stumbling dystopian hack-jobs who lack transparency. Traitors to the “Do No Harm” standard. Disguised as “legitimate medical solutions, doing a high-wire act.” It's a cautionary tale how each dystopian, non-essential device trades profits, above patient care (as subjugated patients rise-up from the front-lines). Another comparison comes down to essential devices, versus non-essential devices during COVID, for family-medicinal purposes.

Comparatively speaking, challenger devices are draconian widow-makers. Absent DCP-D's deep-bench of: AI/AR, cryosurgical dialed-in the zone discretion. Imposters parading “as the real-deal.” Prioritizing profits over quality-care. A careful examination of predicate nomenclature reveals they completely miss “the CAPA imperative.” But what they don't disclose is more startling . . . their offerings are “subject to errors, omissions, change or withdrawal without notice (or ADA/AMA approval).” Human rights abuses. Absent the cache of 3D biomimicry tooling. Failing the Hippocratic Oath of Doing-No-Harm and limited by Neanderthal thinking. A false-flag. Innocent patients between the cross-hairs. Absent is an arsenal of pioneering technology: Panoramic HD Surround-Vision, Broad-shoulders, a Backbone, Edge-Glide-Rails; Spec-Ops/I-OPS accuracy or safety-net of Level-2 bioengineering. Level-2 is a “confederation united by a circumferential, MIT terraced-band.” Several predicate devices, despite being FDA 510(k) approved employ: caustic, rocket-fired, cancer-causing aerosol propellants directly at patients! Inadequate, short-sighted, unequally-yoked, unethical and rather crude. Subjecting patients to “unfounded chemical bombardment.” A Hail-Mary, evasive approach upon besieged patrons. Failing customized patientcare. Falling-short of DCP-D's: safe, security-guarantees and continuity of leadership. Absent are DCP-D's full-service Spec-Ops-7 (short-range strategic arsenal of 7 UHD/IMPs: 2, 3, 4, 5, 6, 7, 8 mm). A stakeholder controlled “petcock effect” where specific lesions are trained-on Sweet Spot/Danger Zones: eye-sockets, genitals; public and private “dark-places.” Larger, tactical IMP socket-keys further extend Close Quarter Combat (CQC) range; saving untold human lives.

As a Level-2, GT Unlimited-Class Rx tool, technical triage made easy; DCP-D places lesions in Stage-1 of Suspended-Animation. Moles, tags etc. are in close-quarter-hold. Facilitating a Just-in-Time, Frozen-in-Time frozen tourniquet proposition. Asserting Micro-G downforce, utilizing a multitude of leveraged suspension dynamics. Prepped, pre-staged and clinically pre-positioned for Stage-2 cryodestruction procedures. Over-engineered, it-breaks old rules and overcomes deficiencies of SOP predicate devices. A disruptive family hospital platform designed to Serve-and-Protect patients suffering from benign to aggressive skin-cancers. Placing lesions inside a structured, walled, pre-triage framework. Producing a precision hole-shot through an IMP “Eye of the Tiger” site picture. Locked and loaded; most lesions will die in a decisive, ground-fight shoot-out with DCP-D.

Born from a Krieger-Amulet-TaaSa method, DCP-D is multi-dimensional. Compassionate. Setting new, clinical-rules of engagement. R&D is based on Regulatory Pathway Assessment (RPA). Peer-reviewed and scrutinized under CAPA, cGMP, and pre-emptive best-practices by MDs. Producing traceability, accountability. Close-Quarter-Hold (CQH); tourniquet auto-locks (like LEGO® blocks); using “aperture-suppressors” formed around naturally occurring lesion-shapes. IMPS act as Braille picture-puzzle-pieces (the Braille-effect). Redundancy. Fast user set-up. Quick-draw transition. Able to circle-back to re-treat any nagging lesion. A clean, tactical, operational routine. DCP-D incorporates “Halos”. High-Altitude, Low-Opening IMPs. IMPs are purpose-built, data-points. 3D engineered. Workflow optimized. Leveraged upon an ability to delve into tight, outlying areas. Operational logistics. A physician-extender. Quality-care replete with AI/AR safe guards. Sufficient deterrence's against lesion regrowth (recidivism).

Artisan-styled. A single-unit, sled-like device. Skeletal-Girder-Kinetic-Exoframe chassis produces the Braille-effect. A nomenclature designed specifically for sub-zero use/reuse under UV/IR clinical autoclave sanitation protocols. Medical-grade materials having exceptional: moisture, chemical, virus-barriers (CDC attributes). ADV-01/DCP-D incorporates cGMP durable Nano-tech jacketed-coatings or layering composites. Stakeholders may autoclave & sanitize DCP-D enumerable times, without degradation to device. Adding value to the supply chain. DCP-D is sustainable. A green décor product. A unique, crafted blend of Ui/Ux inspired Med Tech. Carbon-neutral. Humanitarian values.

Outwardly-biased winglets gently pry and hold open skin-folds during Stages 1 & 2 cryotherapy. “If a safer, superior technology exists” (the argument goes), “Are dermatologist-providers ethically duty-bound to explore using DCP-D?”

DCP-D glides. It surfs. It floats and skims across the skin, effortlessly. Defying SOPs and the laws of gravity, using downforce-ergonomics and precision; incorporating a plurality of Under-Body Edge-Glide Rails that pries and holds-open sweaty, hairy skin-folds. Maintaining “track-position” inside tense, uncertain situations without slipping or fowling triage. CAPA-precision hole-shot accuracy. Leveraging-in preset, molded-in Spec-Ops-7 cluster. Conforming lesions; transforming tissue. DCP-D sticks the landing without substitution or dilution.

Using MIT/IMP-keys circumferentially conforms nevus and tags; profiling lesions. MIT is a design-canvas characterized as a Level-2 terraced perimeter band “teocalliesque”. Thicker than a majority of skeletal Exoframe chassis (slide-polymer frame). Having no IVD needles, no balloon cannulas, biopsies, old-school otoscope speculum; or antiquated SOP's. Closing porous-borders. IMP's are cryofire walls, able to focus, target and choke-off lesions. Building walls. Encircling. Safe gain-of-function destruction. Taking-out the guesswork. Consistent. Repeatable.

Of note, a Next Gen 2.0 improvement over standard holographic otoscope; funneled specula of old. Augmented Reality (AR), Artificial Intelligence (AI) interfaces, producing Rapid Triage Elements. Ground-zero, optimized, binary, user-interface index. By regulation DCP-D fits squarely into a specific FDA field of . . . “General Surgical Tools and Accessories.”

A design-canvas-matrix, Holographic Augmented Reality (HAR); in-situ lesion containment system. An easily mastered Rx skill-set.

III. End Goal of Med Tech Device

Shown herein is “a manner and process” of making & using DCP-D . . . “in such full, clear, concise, and exact terms as to enable any person skilled in the art to which it pertains (or with which it is most nearly connected), to make and use the same, and shall set forth the best mode contemplated by this sole inventor; Rory Krieger. For purposes of discovery, inventor and stakeholder addresses “common lesions found growing on healthy-normal-people.”Many patients suffer with Stage-4 skin cancer; where some are also prime-time, out-patient candidates for tailor-made, personalized treatments. To that end, DCP-D does not address a myriad of dreadful, exotic, oncological or zoological aberrations that also exist. Nor indulge the twisted circus-freaks, or a gaggle of case-studies ripped from the pages of “Ripley's Believe it or Not!” DCP-D and ADV-0l are identical.

Pocket, lab-coat sized Form-Factor (FF), ADV-01 requires a combination biologic cryogen means, to perform its duties. Favoring the cryo-liquid LN2 (as preferred biologic of choice); executing the “metered hole-shot.” Judiciously applied LN2 will adequately effect the desired result; necrosis. “Off-grid” this device is not AC/DC, or battery powered. DCP-D “co-pilots the user” to: rotate, gimbal, pitch/twist, cross-thread, crab-walk, straddle, back-side rodeo; Half-pipe 180° Swing-set, and moon-walk across a patient's skin. Skill-based triage. Taking ownership of On-Demand Cryotherapy. Human Factor (HF) simplicity-by-design. Dialed-in, purpose-built, COVID-compliant. Humanity served by a single-unit Ui/Ux empowered primary-device.

3D Holographic AI, with gain-of-function. “Zero-G micro-gravity” redundancy; Just-In-Time (JIT) with unblemished symbiotic best-practices. Taking triage to the edge of simplicity. Add it all up and DCP-D (ADV-01) achieves . . . “non-obvious material to patentability of the invention claimed in this non-provisional utility application.” Hardness of frozen tissue is critical in triage; and a key consideration for depth of cryogenic therapeutic treatments and dermabrasion. ADA classified range: superficial (0.2-0.5 mm), moderate (0.5-1.0 mm), or deep (1.5-2.0 mm). DCP-D utilizes a safety-gantry halfpipe design for quick transition. Substantially protecting patient from an LN2 spill.

### DETAILED DESCRIPTION OF INVENTION

DCP-D Wand is FDA designated as a non-active. A stand-alone device (with no constituent parts). DCP-D (ADV-01) does not require electricity, radiation, LEDS, needles, cannulas, tubes, etc. Not FDA designated as: IVD (Class-2 or Class-3).

DCP-D mandates use of: a biologic Combination means: direct LN2, cryopens, cryogun, mini-cryogun; pressurized spray-on Freon gas, etc.). DCP-D also interfaces with non-biologics: cryopen/probes.

The biologic allows DCP-D to fully produce destructive Stage-2 cryogenic necrosis (when applied carefully). A de-risking tool for LMI's contain: molded-in mosaic-socket patterns. Angled, canted, Multi Factor Authentication organic aperture-suppressor IMP-keys with relief under-cuts. Based-upon a carefully-balanced-array of Just-In-Time (JIT), Rapid Triage Elements (RTE); bio-motion recognition. E20-11 (Easy to Operate-11).

B2B Enterprise Collaboration+B2C Beneficiary Stakeholders

A single-source-solution unit without Constituent Parts; DCP-D uses a cryogen means . . . as its only Combination Part, performing cutaneous cryosurgery. Prolonged reagent penetration, with precision targeting and safety. Extending the value-chain, DCP-D has a deep-bench in this dual-vector cottage industry. A safe, proprietary, disruptive, bespoke, workflow-optimized tool. Multiple working volumes. Internal “X-Y-Z counter-balancing.” Molded into a professional-grade, shirt pocket-sized form-factor (FF). Rugged-design. The Total Product Life Cycle (TPLC) of DCP-D/ADV-01 is long-term; multi-use. Shared-mobility compliant; a multi-use Med Tech, with an extended life-span. Sanitize, and reuse it over and over again. A binary user-interface. Should DCP-D break? It's bio-recyclable (green). Environmentally sound.

Doctors and patients know lesion isolation is key. On a case-by-case basis, a patient's trust in their doctor is contingent. With so much to live for, stakeholders know “border-walls around lesions, save-lives!” Use of a novel, disruptive Med Tech, DCP-D fits into: battlefield readiness, medical tourism, OB-GYN, L.G.B.T.Q.I.A.+ friendly. Cosmetic, oncological, cryotherapy device (and the like) designed for general dermatological triage by: LMI's (Licensed Medical Intermediaries), NPP's, conscripts and OTC-DIY home healthcare. Ui/Ux precision designed for high-definition (HD) “Manual surgical instrument for general use” [21 CFR Parts 862-892].

For B2C home healthcare markets, the same methodologies apply. Each market segment uses the same 2-Step process. Treating the same broad-range of common epidermal pathologies, plus skin disorders including: A.K., acrochordon, basal-cell, squamous cell carcinoma (SCC), cancerous lesions, cysts, flat-warts, keloids, lentigo, mucocle cysts (wart clusters), moles, morphologies, peduncle-hanging warts, S.K. and many other types. Employing a Human Factor (HF) technology that is intuitive. DCP-D is packed with feature-rich nomenclature that no “prior art device” offers. “Animated, dancing across-the-skin. A coordinated, surgical-attack. Designed and built for shrewd stakeholders who demand more of themselves and their patients.”

Identifying Epithelial Abnormalities

Lesions are treated on the outer-epidermal (visual surface-layer) of viable living tissue. As such, DCP-D does not take biopsies, nor use of radiation, MRI, IVD needles or IVD balloon-catheters. DCP-D is non-obvious (horse of a different mother). Clean, green; sustainable, responsible. Surpassing the reckless futility of each prior art predicate. A “witches-brew of trap-door devices” targeting naïve, trusting-patients (and the shills who promote their Trojan-horse iterations). The gaggle of contraptions favoring profits over patient-care; in their apparent scorched-earth model and callously dismissive post-op lack of accountability. DCP-D is patently different! Below is a partial-itemized listing of common skin-lesions (primary morphologies) that DCP-D is Turn-Key Ready to safely terminate.

Indications of Use (Commonly Treated in General Practice):
Acrochordon (small, soft, skin tag growths)
Acne Surgery
Actinic Keratosis (AK)
Age spots (Lentigines, Lentigos or liver spots) and skin growths
Anal skin tags/fissures
Angiomas
Acne scars and keloid repairs
Cavernous hemaglomas
Common Flat Warts: rough discolored & “cauliflower-like”
Condyloma Accuminatum; genital warts (EGW-HPV)
Cysts
Dermatafibromas, small hemaglomas
Dermoscopy (pigmented skin lesions)
Excisions
Flat wart destruction
Hemorrhoids
Keloids [scars] (freeze-time 30-90 seconds on average)
Lentigo (freeze 20-30 seconds)
Mucocle cysts, multiple wart clusters (eye sockets) face, neck, thighs, etc.
Moles (average freeze time 30-60 seconds)
Molluscum Contagiosum (freeze time 3-5 seconds
Non-recurrent basal cell (freeze time 90-180 seconds)
Papilloma (skin tags)
Plantar warts (bottom of the foot; Freeze 60-90 seconds)
Proctology
Peduncle Skin Tags (protrusion of a tag—hanging finger)
Recurrent cancerous tumors
Seborrheic Keratoses (SK)
Skin-Tag Clusters (acrochordons) are exceedingly common. Often found in clusters (freeze time: 30-90 seconds).
Warts/plantar warts (freeze time 45-60 seconds) and benign pigmented, epidermal and cutaneous lesions. Including common warts, skin tags, tumors, cancer etc.
Verrucas (Verruca Plana, Verrucas Plantaris, Verruca Vulgaris) are very similar to common warts but mainly appear on the sole of the foot, heels or toes and tend to be much flatter in appearance, sometimes as callous skin.

DCP-D (ADV-01) also treats stratum corneum epidermidis, and miscellaneous solid tumors (per otoscope speculum cryotherapy). Having the added benefit of a proprietary Level-2 Halo-Deck Banded-MIT Corona. Tiered-Band is thicker. Useful as a biologic isolate; further adding to the speculum otoscope 2.0 funnel-method for more accurate triage procedure. Adroit for device lite-weighting. A design paradigm supported by STEAM technology. FDA EUA; SARS-CoV-2. Pandemic-Deployment Compliant (P-DC).

Providing a verifiable and repeatable mixed-reality methodology; holographic AI shielding for non-effected virgin tissue during “flash-freeze” Stage-2. Preventing cosmetic collateral damage.

Achieving HD performance commensurate to board-certified LMIs, MDs, NPPs, conscripts and OTC DIY practitioners. Along with zoological veterinaries and early-adopters will greatly benefit from this “Copilot 360° health tech revolution.” Substituting-in and favoring a Discrete Cryosurgical Primary-Device. As it reevaluates the current gaggle of antiquated, copy-cat, medieval gadgets that are embedded (and senselessly linger) in this cottage industry. Predicate prior art has a tendency to disparage physician reputations during a pandemic. Where patients miss-out on daring Elective Surgery procedures, and humanity itself is unfairly held-back from holistic living.

### V. DESCRIPTION OF DRAWINGS

Referring to specification figures, images clearly delineates the nomenclature of DCP-D (with respect to FDA Regs. 37 CFR 1):

FIG. 1 shows a “Topside Profile Length” of DCP-D 3D Wand-base Halo-Deck (front-page shading view).

FIG. 2 shows an “Elevated Topside Prospective View” of Wand-base chassis (as a solid-model, without depicting detailed MIT terrace, or IMP-key mosaic apertures).

FIG. 3 shows a “Top Profile Length Solid View” of DCP-D Level-2 3D Wand-base (absent are IMP-aperture details).

FIG. 4 shows “Under-Body Profile View” showing the Backbone-SRS. Note: Under-Body Edge Glide-Rails not shown).

FIG. 5 shows a “Distal Upper ⅓ Partial Scale-View” of Wand; multiple IMP-key protocol apertures. Note: zoomed-in MIT/IMP sockets and features are omitted in the following FIGS. 1,2,3,4,8,10,11 to avoid confusion, minutia obscurity.

FIG. 6 shows a “Proximal Tail Sectional View” of DCP-D 3D Wand-base, at bent tail-section where IMPs narrow and widens providing slide-in and crimp sockets. Facilitating next-generation strategic, tactical-operational protocols. Note: zoomed-in MIT/IMP socket-key halo features shown here, were omitted from FIGS. 1,2,3,4,8,10,11.

FIG. 7 shows a “Top Length Wand-base View” of DCP-D 3D Wand with MIT/IMPs molded in Level-2 Halo-Deck Band. MIT/IMP features are omitted in FIGS. 1,2,3,4,8,10,11.

FIG. 8 shows a “Bisecting Mid-line Undercut Groove CAD Sectional View” (mid-ridge groove) of Wand.

FIG. 9 shows a “cross-section depiction of Stages 1 & 2 View” of Level-2 MIT Halo-Corona-Gap (IMP-keys), as Stage-2 cryotreatment occurs to an epidermal lesion.

FIGS. 10, 11 spotlight ADA (American Dermatology Assoc.) method-spray-patterns; including industry accepted propagation/penetration through an IMP-aperture socket-key.

### VI. INDEXED NOMENCLATURE OF DCP-D

8—3D Halo-Deck Wand-base Exoframe perspective view [Top-Side]
9—3D Halo-Deck Wand-base Exoframe perspective [Under-Body]
10—Arcuate Bowl Architecture [anatomic arcuate ergonomics]
11a—Level-2 Halo-Deck Distal-Banded-MIT-Corona [CQH portals]
11b—Level-2 Halo-Deck Proximal-Banded-MIT-Corona [CQH portals]
12—Distal-Head [with Under-Body stabilizing edge-glide-rails]
14—Up-bent Arcuate Neck Segment
15—Spec-Ops-7 IMP Cluster Socket-keys [Metered IMPs]
16—Broad-Shoulder Wing Segment
18—Svelte Waist Segment
20—Hip-Wing Segment
21—Under-Body Edge-Glide-Rails
22—Ankle Segment
23—Tail-Bowl IMP Halos [proprietary protocol-keys]
24—Proximal-Tail Up-bent Segment
25—Tail-Edge Open Halos
26—Under-Body Backbone-SRS [Zero-G, micro-gravity; XYZ-axes]
28a—Distal Linear Arcuate-Bowl Architecture
28b—Proximal Linear Arcuate-Bowl Architecture
29a—Florentine Styled Elongated Aperture [proprietary IMP]
29b—Florentine Styled Elongated Aperture [proprietary IMP]
30a—Under-Body Distal Matrix
30b—Under-Body Proximal Matrix
32—Bisecting Mid-Ridge Groove
34a—MIT/IMP Distal Matrix [metered-socket protocol-keys]
34b—MIT/IMP Proximal Matrix [metered-socket protocol-keys]

[DISCLAIMER: Throughout specification of DCP-D, the 3D Level-2 Halo-Deck Wand-base (8) is hand-carried. A mobile, reflexive, bendable, sled-like tool for cryotherapy. An artisan-styled, digital decoded-encoded, Human-Factor (HF) chassis-platform. Enumerated-in svelte “bucchero” human and military terms. Distal-Head, Broad-Shoulder, Hip and Tail, Backbone-SRS segments offers a 360° continuous, circumferential open-halo outer perimeter feature. Chamfered and beveled-edges for insertion into tight, hard-to-reach areas. Bilateral, aperture metered-socket protocol-keys are a subset to said MIT Level-2 terraced band. Circumferential with respect to Reflex-Wand. A digitally encoded-filter membrane that shape-shifts a lesion, thru a “decoded” IMP socket-key filter. IMP protocol-key (socket-fixtures) execute a “choke-suppressor” function. Furthermore, Arcuate Bowl (10) of flexible, on-axes, 3D Kinetic Exoframe Wand has substantial lite-weighting; agile, rigid, gimbaled, anatomic bio-recognition auto-lock; CACHE-IN-AOD capture-feature over lesions/tumors/etc. Self-indexing aperture metered-socket protocol-keys provide Close-Quarter-Hold (CQH), Tourniquet-Capture throughout the 3D Halo-Deck Band. Spec-Ops-7 “munition IMP cluster-keys” (15) provide fine-tuning capture for 1:1, 2:1, 3:1 triage. Florentine apertures further extend user-reach of tool. Select IMPs contain gradient-sized Star-Ratio Patterns. IMPs are positive gain-of-function (termination) assets; funnel-chokes with undercuts, operating under “a Braille-effect” feedback loop. Combined, proprietary IMP's enhance protocol-portals to produce an integrated First-Responder-Asset as a “Mobile Strike Group” device; utilizing Rapid Defense-Rapid Offense Procedures™ (RD-ROP). Under-Body Backbone-SRS is ballast; ballast with rails (surfing-along its slide-polymer frame; facilitating a Just-in-Time (JIT), Frozen-in-Time (FIT); Lesion Harvesting Strategy (LHS). A Top-Sided Stakeholder-Draft Guidance (TS-DG) which teaches “Intel Surveillance Recon” (ISR) best-practices; Web link menu supports stakeholders as a co-pilot. Aka, an “incubator knowledge accelerator” for newbies entering cryotherapy, and seasoned pros still practicing cryosurgery. In all, codifying remittances and reimbursements, backed by Medicare/Medicaid [CMS.gov.].

Under-Body (9) of 3D Wand-base (8) exposes central Backbone-SRS (26). A tool with a backbone for shared-mobility, quick-triage; controlled-gliding, pitch/twist, gimbal. Under-Body Backbone-SRS-prevents snags. Improved triage results. Not shown is a Under-Body Edge-Glide-Rails (21) in pivotal areas. Allowing user to control the glide-slope; in multi-directional gyrations (straight/linear, crab-walk, cross-thread, moon-walk motions). Useful during surveying Stage-1 exams, diagnostics. Snapping-turns, 360° rotations; circling-back for retreatments.

In the spirit of reciprocity and reconciliation, tool serves the public-good by democratizing quality-cryotherapy. Level-2 Halo-Deck Banded-MIT-Corona (11 a/b) provides Wand with a thicker/deeper Wand funnel aperture-socket, within the Level-2 MIT halo-band matrix. Deeper aperture facilitates a longer cryogen penetration-cycle over sensitive lesions, while reducing circle-back sorties and costly patient-revisits (CAPA-oriented). Level-2 MIT (with subset IMPs) represents “a thicker-banded area around an IMP Halo band-deck” of Wand. IMP aperture-sockets are integral and adaptable-components. Scaled-models that require forethought and skill by the user. Tilted and rolled Close Quarter Hold (CQH), thanks to a Backbone-SRS central-glide point. Whereas the Under-Body Glide-Rails (16) provide lift, probe and linear stability. Preventing skin-fold slippage. IMP's lesion-size variable. Holding a clinical range of CQH, able to auto-lock, self-index over cauliflower, malformed lesions. Gravity-defying, floating-ballast. High flexible downforce allows device to achieve the proper attitude and point-of-contact with ambient temperatures. It's a question of balance; dexterity-fidelity-efficiency. Providing a unique 24/7 clinical bump-feedback loop. A Bisecting Mid-Ridge Groove (32) substantially surrounds perimeter of Wand-base (8). Under-Body Backbone-SRS (26) eases maneuvers during Stage-1 lesion sorties.

Moreover, SRS acts as a Snag Reduction System, allowing smooth gliding (skin surfing) without the risk of cutting hair, fleshy-tissues. Policing the zone for suspect lesions. Floating like a butterfly; having an “Airbnb-quality” with SecureFIT, auto-lock mitigation strategy. Hence, Glide-by-Wire™. A precise glide-slope for HD patient-care. Under-Body Edge-Glide-Rails, like rudders on a boat, guides tool where it needs to go; prying-open skin-folds (strategic leverage). A tactical operation; picking-off small nagging lesions one at a time. Aiding in users panoramic, 360° and quick-site picture. CAPA compliance. DCP-D provides a precise 100:1 kill-ratio. Multitasking Assets on Demand (CACHE-IN-AOD). Skeletal-Girder Reflex Exoframe Wand was bioengineered for Offensive-Defensive systems. Navigating and sliding the skeletal chassis ever-so-gently. Patrolling, gliding the visible epidermis. Surveying, probing, always on the look-out for suspect tissues. Intervention, capture and evisceration of unauthorized bodies. Diffusing tensions between healthcare providers, and innocent patients.

Topside Level-2 Halo-Deck Banded-Corona (11a, 11b);
1. Simply put, the halo-deck band marks “a thicker topside Wand surface” encompassing all interior IMPS; allowing for deeper-penetration, yet safer-pooling of LN2 (qualifying cryogens and cyroprobes, etc.). Allowing for a mechanical capture. Aperture-silo barricade through extended-exposure in the drop-zone. Think “aerial drone-strike” by a cryogen. Directed by Spec-Ops-7 guided IMP munitions. Offering a degree of added safety-shielding (CAPA); as Wand (8) is rapidly repositioned over tiny (or large) affected lesion(s). Crucial for sensitive eyelid hanging peduncle lesions, and around the eye sockets where curettes often grow as finger-like, dark fleshy grotesque spores and clusters. Facial areas, breast nipples, cosmetic; HPV-related infections. AIDS, VD-prone private genitalia. IMP undercuts in bottom-side sockets prevent LN2 from leaking. Forming a sealed-demarcation of safety around each lesion. Supported by: CAPA, MIPS, MACRA; fiduciary interests.
2. Discrete LN2-pooling, facilitates extended cryogen exposure, depth, penetration, and necrosis efficiency. Regulatory Pathway Assessment (RPA) engineered. Including B2C home-healthcare by the general-public. Pre-positioned. Angling for the highest fidelity possible; and for the highest degree of safety. Highly-sensitive areas, require Spec-Ops-7 cluster. Specialized aperture-sockets keys used in sensitive areas (under the steadiest level-of-care). Incremental in size for delicate, discrete cryotherapy.
3. Each aperture key-socket operates independently. Needless. Non-IVD. Non-invasive; Self-Indexing Compensators (SICs). Gently forcing each fleshy lesion/morphology/tumor to adapt and conform to a “pre-tuned” IMP-key; but for Biomimicing Transformer Recognition (BTR). Hybrid triage with auto-lock retention. Shared-mobility among stakeholders. Operational logistics. Spec-Ops-7 gradient-vectors. Tactical I-OPS. Anti-lesion, 360° cryodestruction. 24/7 triage-ready.
4. Therefore, holographic inspired halos that surround exterior IMPs, accurately pools LN2. Unrestricted, full spectrum flexibility. Shields patients from harmful spills.

In short, Wand-base (8) and Level-2 Halo-Band (11a, 11b) extends cryogen penetration deeper for a “leave no lesion behind” level of care. IMP aperture-keys are permanent, molded-in (not added-on nor constituent parts). Entire skeletal Exoframe Wand-base (8) is completely devoid of “FDA Constituent Parts.” An important distinction to make, since nothing can break-off, shred or injure a patient during simple, out-patient triage or cryosurgical procedures. Rigid, flexible, 3D Halo-Deck Wand-base chassis (8) is resilient. Having a substantially profiled terrace surface finish; compatible for an “Rx label cover” (ISO 15223-1:2021 compliant). An integral strategy for FDA stewards.

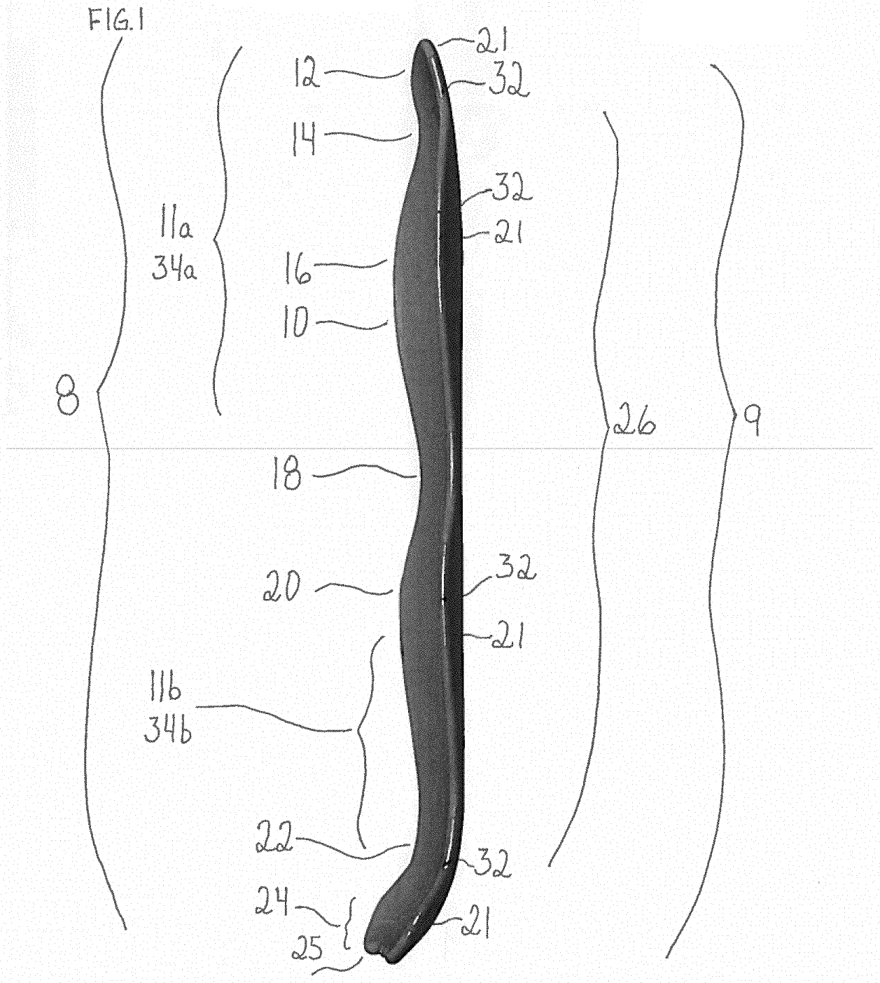

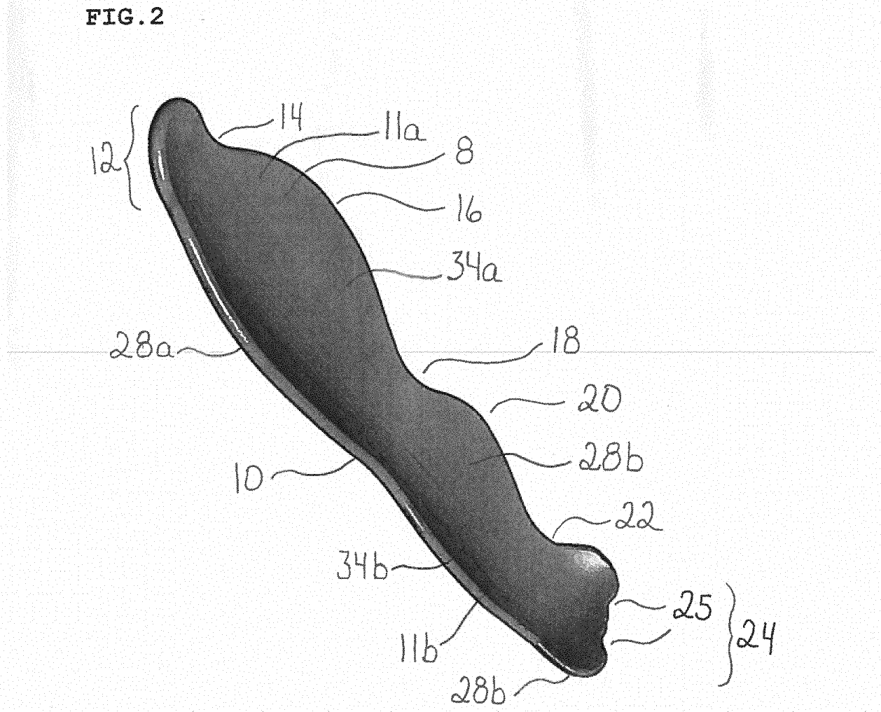

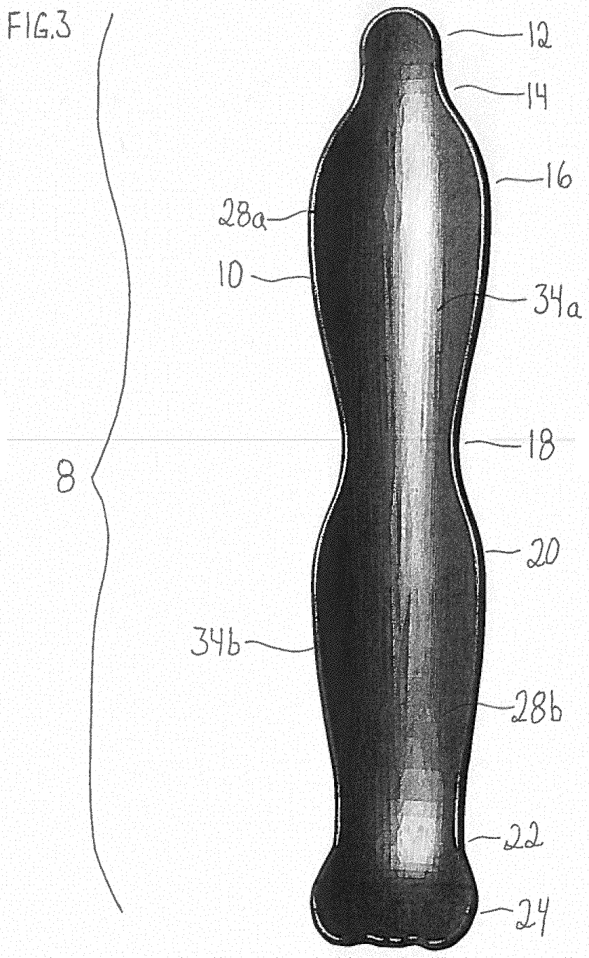

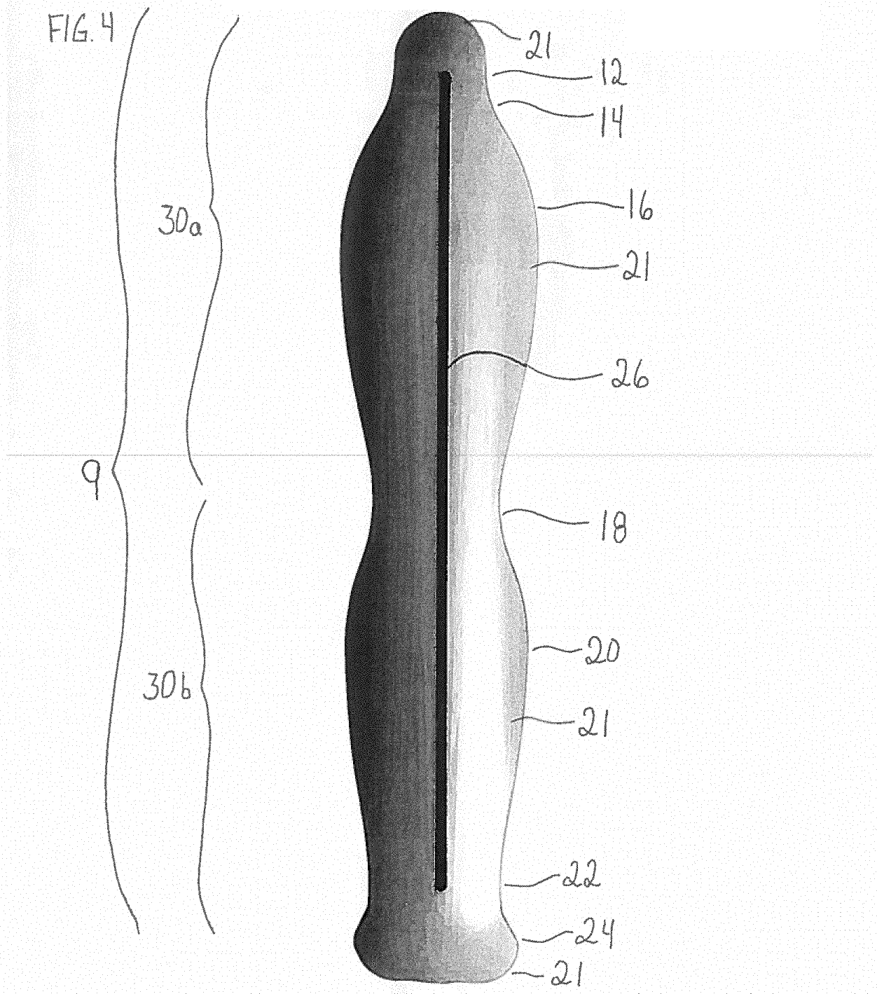

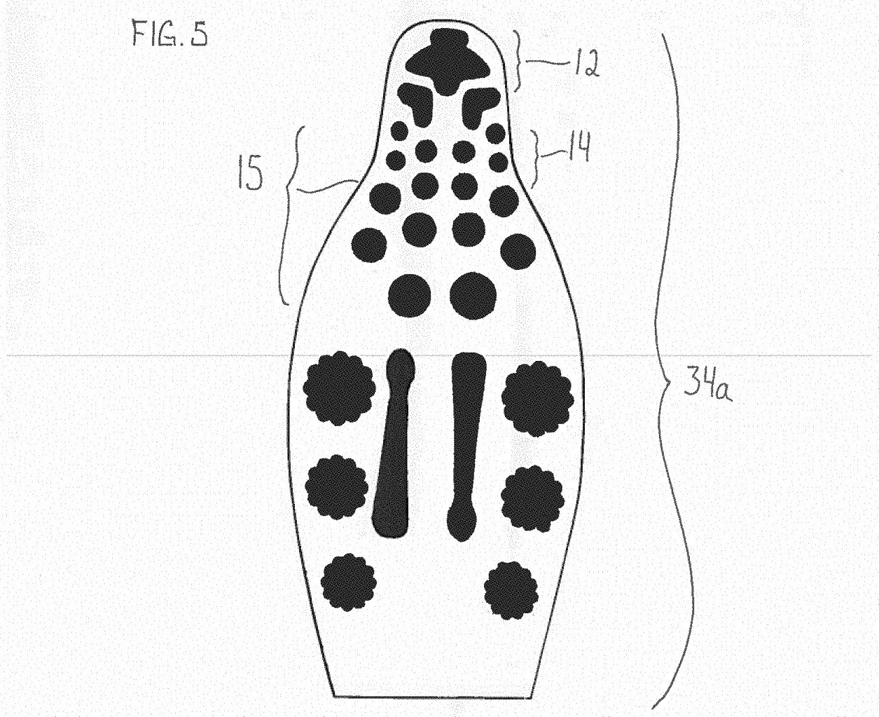

### VI. IN-DEPTH NOMENCLATURE OF FIGURES

[FIG. 1] “Vertical Right-sided Perspective View” shows a solid, Reflex, Exoframe Wand-base (8) chassis (DCP-D). MIT nor bucchero IMP's are shown in this drawing (allowing for ease of viewing device; as well as its svelte, sled-like architecture).

[FIG. 2] “Perspective Profile Length Solid View” of wand shows dished-out, concave, “shoe-horn appearing” architecture of Wand-base chassis (8). Moving down from head to tail, neck, Broad-Shoulder Wing Segment; Svelte Waist, Hip-wing Segment and full-compliment of half-pipe, Reflex, Exoframe Wand chassis (8).

[FIG. 3] “Level-2 Straight-on View” is shown. MIT and IMPs are not shown, nor the bucchero nomenclature. Allowing examiner to flesh-out % pipe, stadium landscape, allowing for a Panoramic, and HD Surround view of the arcuate, elongate, sled-like Wand.

[FIG. 4] “Under-Body Chassis Profile View” of skeletal Wand. Image shows Under-Body (30a/30b) distal matrix form factor is convex (like a sled rail). Running down-along-center of DCP-D is the Backbone-SRS (26). Backbone-SRS “spine” is smooth/polished. Off-set with respect to depth/width (XYZ-axes). Having a rounded “half-dome orientation” protruding along Wand bottom. Intended purpose is for tilt & ablate rapid-gliding. Lean-to, lean-for tunicate capture of a lesion from the central SRS pivot-point. Edge-Glide-Rails facilitate constant contact with the skin while gliding-over bumpy skin during Stage-1 exam. SRS also signals a feedback-loop to caregiver. Proactively indicating to user, the faintest/flattest lesion encounters.

[FIG. 4] Also shows Under-Body Backbone-SRS (26) of Exoframe Wand. Absent are Under-Body Edge-Glide-Rails (21). Rails provide tracking and control. Allowing Wand-based glide-slope surveying. Turn-key enabled in strategic areas. Rails can press, push and pry-open skin-folds for effective, non-invasive triage.

[FIG. 5] Shows “Distal Upper ⅓ View” of Wand-base (8). A busy image for sure, much is shown including: Level-2 band, MIT/IMP allowing pitch/twist, yaw & roll” for 3D action. AR-controlled aperture-keys enabling over-the-horizon capabilities. Oriented distal to proximal; IMPs go through Wand. Where a particular IMP contains a halo-gap, Level-2 (terrace) also provides a halo. FYI: Tail-Edge Open halos are deemed “unlocked” (not enclosed).

Close-up view shows individual Spec-Ops-7 cluster-keys (15). “Numbered & Nesting Apertures” at tip & neck-segment close to the Wand's center-line. Spec-Ops-7 cluster is a tactical AI/AR feature. Useful during sensitive triage on: the face, eye-lids, eye-sockets, genitals, etc. Maximizing a 360° Lesion Masked-Mandate Rule. A full-suite Spec-Ops-7 gradient (IMP socket-keys) ranging in millimeters as: 2, 3, 4, 5, 6, 7, 8 mm; employing a digital-twin (left/right) decoder as a match-grade MIT toolset. Providing one example of “Interdiction-Consumer-Empowerment” (ICE) protection for patients (a mechanical insurance policy).

(NOTE: As a 360° user-toolbox, Skeletal-Girder Reflex Exoframe is heavily engineered, yet lite-weighted. A skills-based, technician's tool using familiar, svelte, human-like features under wartime terminology. In FIGS. 5-6, note how the zoomed-in MIT/IMP sockets, decoder/encoder features are conspicuously absent in: FIGS. 1,2,3,4,8,10,11. Intended to unclutter, clean-up reduce confusion of Wand as a full-service, asset on-a-stick.

IMP-sockets are clinically bio-engineered to probe/navigate, intercept and auto-lock (ShureFit) on to a wide-range of epithelial morphologies with impunity. ShureFit “decodes and transforms” a lesion to a corresponding aperture socket-key, for an accurate, leak-proof clinical-fit. STEAM-designed for: 360° probing between toes, inside the naval, behind ears, skin-folds and other “parts unknown.” Cherry-red angiomas and challenging, hard to reach eyelid canthus tags notwithstanding.

Whether the end-user is a board-certified physician, an NPP home healthcare provider, conscript or self-treating DIY patient. SOP's for Stages 1 and 2 still apply. Where top-side label provides a web-based guidance resource to learn best practices.

3-Stage Method of Use: How does DCP-D Interface for Cryosurgery?

STAGE-1 End-to-end asymmetrical triage (one-hand needed). User/physician will glide Wand-Base (8) carefully over the epidermis; evaluating & scrutinizing suspect growths on-the-go. Monitoring, examining common skin lesions “ID-foreplay with glide-rail feedback.” Key match-ups; surfing the epidermis “ISO a mate.” Squeezing lesions into an IMP (auto-lock). Up-close and personal. IMPs provide a unique, over-the-top HD site picture. Whereas, Backbone-SRS will bump into everything protruding.

German-engineering, made in the USA; allows for a “mix-and-match auto-lock test-fit method” where user applies a measured downforce of pressure. A basic-tenant that traps and seals pre-selected lesions (or lesion cluster) “inside a box”. Allowing user to determine: phenotype, size, color and histology. Isolating a lesion; coming-in tight on-entry. Capturing and holding it inside an IMP socket in a snug, pre-ablation manner. Providing a Zero-G micro-gravity XYZ capture; within striking-distance to deliver LN2 ordinance. A holding-pattern. Sheltered-in-place. Targeted. Conforming to biomimicry socket protocols; all of this forms the basis of key-matchups. Pre-stage complete.

STAGE-2: Pre-staged, interfaced; everything's locked and loaded. User/caregiver now applies any number of qualifying cryogenic means; I.e. Q-tip soaked in LN2, a cryoprobe (CRYO2), cryopen, a Freon-sprayer; nitrous oxide pressurized gas means, etc. A judiciously applied “combination biologic” interacts.

A friendly gain-of-function occurs “in the drop-zone” aperture-socket key. Providing a commanding view of lesions, sheltered-in-place. Now it's time to pour-on the coals. Stage-2 requires all-hands-on-deck or an assistant administering cryogen means, as Wand-base tool-handler holds DCP-D steady (if necessary). Most providers can perform triage single-handedly. E20-11 (Easy to Operate-11). Patient and provider both are anticipating the cold-fusion, kill-shot.

With said lesion(s) snugly-isolated inside a pre-selected IMP socket, this marks the one-two stage process of ablation. Consummating the interface procedure: triple-aim, work flow optimized, physician extender, Stage-1 set-up. Worth noting, Spec-Ops-7 IMP cluster-keys (15) are carefully spaced-apart to optimize dexterity, triage placement, virgin tissue masking in order that the stakeholder can make tactical, surgical cryogen strikes, without overfilling the socket, or inadvertently administering collateral damage to surrounding virgin (unaffected) tissue(s). Mission specific, Spec-Op-7/I-OPS keys achieves a high-definition of granular dexterity/discretion. Intended for high-risk areas: face, eye-sockets and private-parts. Expertly used under LN2 timed-pooling procedures. As a tool, DCP-D sticks-the-landing as a Dermatological Pocket-Sherpa. Employing the use of Intel-Surveillance Reconnaissance (ISR) found in the DCP-D Draft Guidance. Thus, a custodian reflex-wand performing triage, backed by a Regulatory Pathway Assessment (RPA), and the ABC's how to perform triage properly.

STAGE-3 Lesion Mitigation Strategy: 2 Bio-Mechanical+1 Natural

Stage-3 is the result of proper alignment, execution and skills based application at Stages 1 and 2. Whereby tissue from normal individuals, will undergo a “regeneration process.”

The ability of skin to heal; re-epithelization of partial thickness burns. Ensured by keratinocyte migration from skin dermal appendages; which begins within a few hours of the cryodestruction. Moreover, the regulatory basis for Stage-3 Blitz-Krieg regeneration cannot be overstated. Clinical accuracy, achieves the DCP-D end-game hypothesis. Significantly eliminating user-error (under strict protocol conditions per ISR Draft-Guidance; ADA/AMA guidelines). Any “mistake” in this area can be costly, injurious, blinding or worse. Save for our ingenious, safe, pin-point, bio-cryoshield design (compliant to FDA/CDRH industry best-practices).

In the event of a Stage-2 accidental LN2 spill, DCP-D uses a CAPA inspired “safety-gantry half-pipe flick-escape procedure.” A rolling-wave design method (per Krieger-Amulet-TaaS™) DCP-D is able to ferry-away an accidental liquid LN2-spill.

This is done by flicking Wand chassis, away from the patient in an instant! Marking device as a user-friendly, CAPA-capable, compassionate first responder. BTW: red-cherry-angiomas are a “specialty” of Spec-Ops-7 IMP cluster-keys (15) with respect not only to angiomas, but also to multitudes of small and large skin-morphologies, etc. Also known as “senile angiomas” and “Campbell de Morgan spots,” red-cherry angioma sized growths were specifically factored-in to the DCP-D Spec-Ops-7 model.

Decisive, capable; “armed to the teeth” with a wide-range of spatially-acclimated IMP socket-keys; ADV-01 creates access into tight-areas. Resulting in a 100% kill-ratio surgical strike. Astute. Uncompromising accuracy. A forward deployed, daisy-chain orientation; preventing collateral damage. That's the drill.

Furthermore, Halo-corona (gaps) are integral, specialized in their own right. Performing a chameleon-quality, DCP-D halo-coronas are bespoke Spec-Ops-7, I-OPS containment sites. A fine-tuning “triage by-the-numbers” approach. Spec-Ops-7 Cluster (15) creates a tight “myopic bubble” to encapsulate. Isolating, guarding lesions inside each metered IMP socket-key perimeter. Handled tactfully, DCP-D guarantees outside virgin tissue is safely protected (inside the bubble) from harsh biologic leaks or cryoprobe slip-ups. Especially useful if an LN2 spill occurs. Preplanning, point-of-attack, execution and withdrawal is one asymmetric, CAPA strategy built-into DCP-D device. A family medicine and mobile-mercenary-approach, providing a “lesion death-trap.” Overcoming numerous limitations, user error(s) and blind-spot failures of prior/predicate art. Thus, DCP-D achieves purposeful ambient temperatures at point of contact, safely. Compliant to: design inputs, outputs, verification; validation.

[FIG. 6] shows a zoomed-in “Proximal Tail Section Up-bent with Specialized IMPs” (24). Several “Stadium 3D half-pipe” IMP-aperture keys (and halo-corona-gaps) are shown (23). Relative to the 3D main skeletal Wand, this proximal tail-section (24) is “lesion-tuned.” Purposely up-bent. Arcuate molded. Using perimeter edges that are substantially “dished, rounded, concave and bio-ornate.” Wherein Tail-Edge Open Halos (25) and Glide-Rails (21) (not shown); are purpose-build for ease of lesion apprehension; ablation in hard-to-reach areas.

Tail-Edge Open Halos (25) are perimeter fixtures located at the “proximal-most, out-edge space” of Wand-Base tail section, and within the Level-2 band. However, Tail-Edge Open Halos (25) are not “locked closed” (versus “open” IMP) halo-corona-gaps. Again, Tail-Edge Open Halos (25) are Open! Open chamfered/beveled. Hybrid, on-demand tooling. Intended to be pushed-up against a lesion (in dark-tight-sweaty, hard-to-reach areas). The entire circumferential outer edge of Wand (8) is beveled, chamfered, and may well be used as a hybrid, unlocked open-halo as needed. Whereas, interior Tail-Bowl IMP Halos (23) are pitch, yaw, tilt, gimbal, twist and roll-centric. All are 3D-control surfaces. Self-indexing, like puzzle-pieces to facilitate AI/AR triage. Furthermore, up-bent Tail Section (24) facilitates unique tactical access. Advantageous for targeting sensitive areas in the groin; skin-folds, toes, etc.; emblematic of Exoframe Wand.

FYI: Predicate/prior art challengers all fail to make provisions for: a Level-2 terrace band, halo-corona-gaps, spatial acclimation Spec-Ops-7. No prior art can offer the deep-bench of proprietary assets, or Cache of Intelligent Novel Assets-On-Demand (CACHE-IN-AOD) nor strategic key-matchups, as those baked-into the DCP-D. Prior art is “Limited and Deficient.”

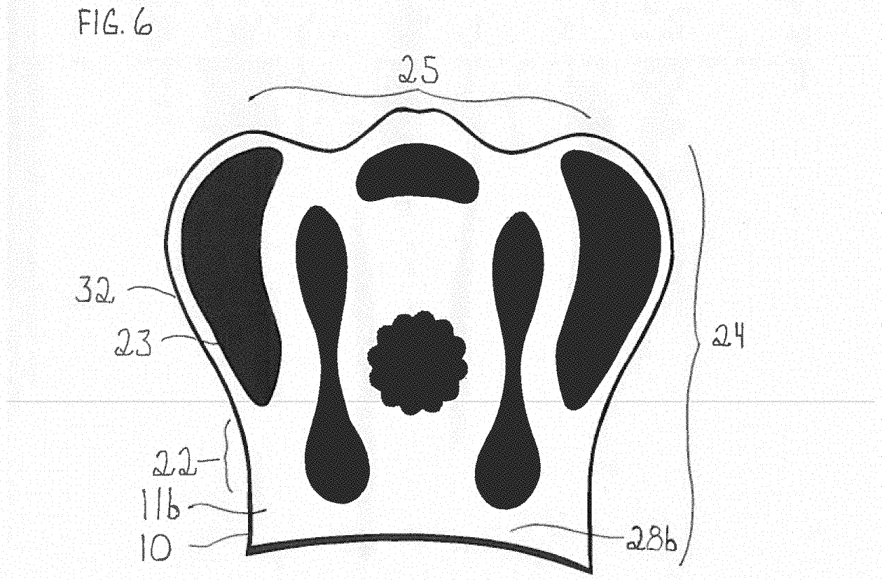

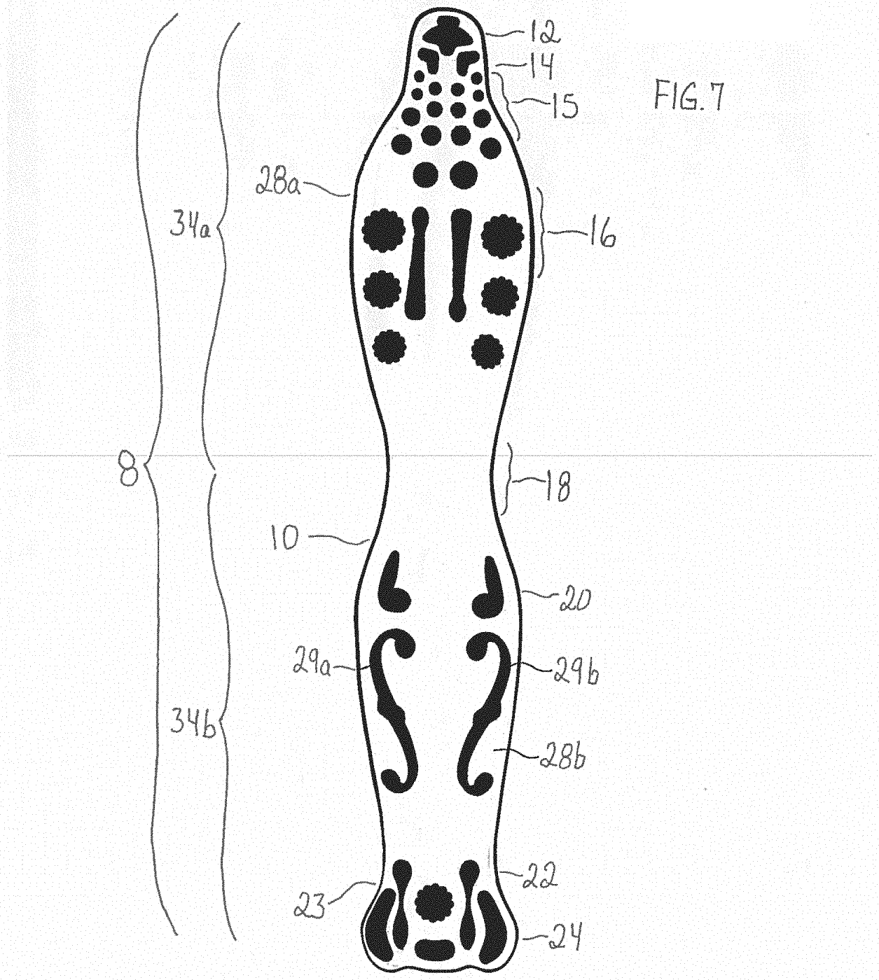

From a CAPA point-of-view, predicate devices are also limited with respect to: Broad-Shoulder Wing Segment (16); Under-Body Backbone-SRS (26); Under-Body Edge-Glide-Rails (21). AWOL when it comes to DCP-D's CACHE-IN-AOD toolset. The depot of pay-it forward DCP-D amenities (replete with 24/7 standby assets).

[FIG. 7] shows “Top Length Aperture View” of 3D Wand. Included is MIT, showing a plethora of digitally-encoded, IMP-key socket apertures throughout Level-2 deck-banded-MIT-corona; from distal end down to the proximal tail. FIG. 7 places most Wand-base nomenclature on full display. IMPs are darkened (as through-hole orifices/apertures) on both left and right mirrored sides. Including the MIT Level-2 (an upper, thicker terrace). This thicker-band contains each IMP (aka “the matrix”). Matrix is the business-end where lesions face Stage-1 compression match-ups; Stag-2 cell cryodestruction. “Multichannel Direct-Input:1 (MDI:1) decodes analog lesion-Inputs, against Digital IMP-encoded Outputs.” Centrally and strategically located across Wand to maximize rapid-triage where lesions/morphologies occur.

In addition (as alluded to earlier); Florentine apertures (29a/29b) is a hybrid variant of IMP protocol-socket-keys. Notice Florentine-styled elongated aperture (29) a/b. This is a proprietary, multi-fit “unlimited-class IMP pairing”. Florentine (29) a/b is considered a work-around at “scripted and unscripted CQH;” striking-a-chord where standard IMPs may be insufficient. A mechanical-hybrid work-around tackles dysmorphic-shaped tissue where other IMP's cannot adequately negotiate. A menagerie of unique, well-positioned IMP-socket-keys are replete in Wand (8).

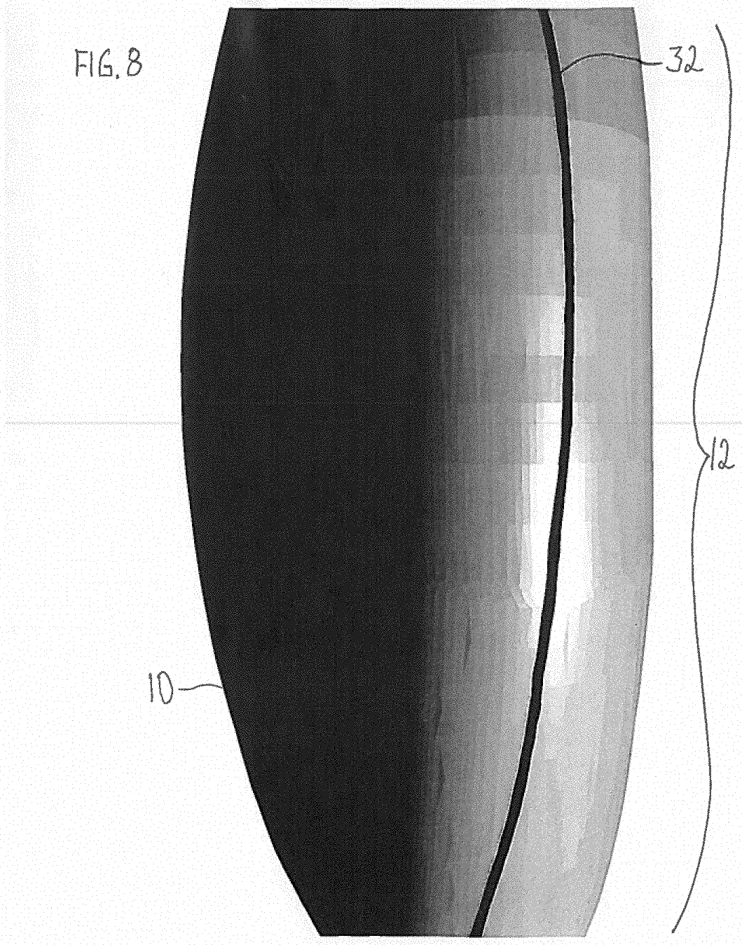

For the record, (8) divisional, derivative iterations of DCP-D exist; including: a). ADV-01, ADV-02, ADV-03; and b). VA-01,02,03,04,05. Where “VA” is for veterinary, derivative uses).

[FIG. 8] shows “Bisecting Mid-Ridge Groove” (32). Designed for Manufacturing (DFM); asset includes molded-in circumferential edging. Positioned equidistant between Level-2 terrace, and under-body. Facilitating hand-grip dexterity; ergo-surveying.

Mid-Ridge Groove creates not one, but two sure-grip, horizontal, circumferential edges for a positive, non-slip grip. Confident, surgical control through latex gloves (ADA/AMA Rx recommended). A medical-device version of AI (driver-assist). Control. Hand eye surgical precision, substantially reducing Stage-2 errors.

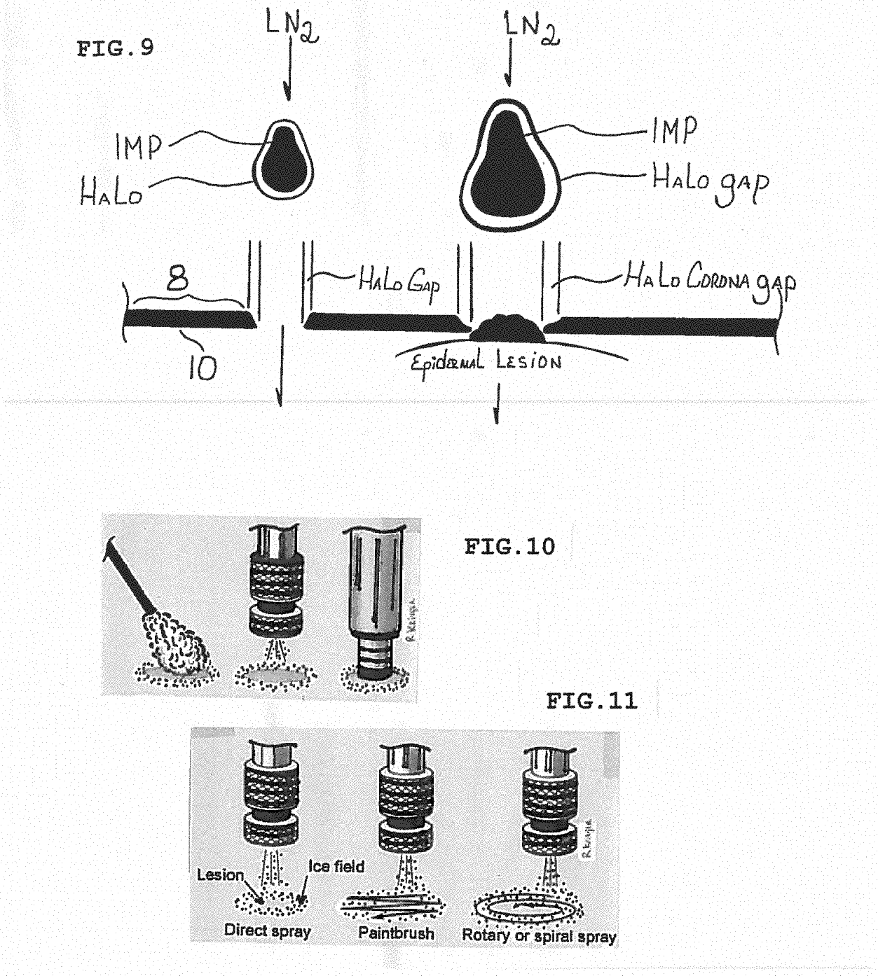

Mid-ridge groove does not interface with Backbone-SRS (26). To that end, Backbone-SRS (26) facilitates surfing, gliding hovering; creating a weightless Zero-G micro-gravity XYZ-axes advantage. A “super-cruise ballast effect.” Light-weighting the glide-slope process while providing a Snag-Reduction-System (SRS). Used in combination with Under-Body Edge-Glide-Rails (21), allowing DCP-D to probe deep-down into skin-folds. Discretely treating a frontier of patient private-parts (with confidence, honor). Providing stakeholders with a “joint-vertical, take-off and landing Braille-effect Med Tech connectivity.” Proprietary. Non-obvious. Uncompromising.

[FIGS. 9-11] illustrates how Stage-2 is conducted. ADA-recommended spray patterns are shown. Distance, inclination, attitude, encoded-filter aperture and timed-spot-freeze ejector-port method (under the “otoscope model”). Visual depiction describes multiple-attack vectors in the DCP-D arsenal; Rapid Defense-Rapid Offense Procedures™ (RD-ROP). Surf, glide, yaw, pitch, pivot, roll and gimbal; a prosthesis-Rx-device exceeding-human-accuracy. AI/AR cache of STEAM, multi-channel filters designed into the TaaS chassis (8). A technician's skill-based, Lifesciences Metaverse First-Responder (LMF-R) platform.

[FIGS. 10-11] show a lesion in the cross-hairs, where a user applies cryogen per: a). Direct-Spray, b.) Paint-brush, c.) Rotary or Spiral-Spray, d.) Q-tip soaked in Liquid Nitrogen (LN2). All are ADA/AMA endorsed. Procedures are administered through an aperture-socket MIT/IMP filter interface-key (open cone shield). Each producing ice-crystals over suspect tissues. Targeted ice-field creates necrosis to a feeder blood-stalk. A timed-shot freeze technique (or micro-burst) inclusive. Either way, user has full-control at protecting/shielding the patient's virgin epidermal skin. Depending on the application approach; judicious use of an LN2 is “prudent and sufficient.”

A careful spray-over application of a “pressurized cryogen” should be administered at 90° and 1.5 cm above target lesion. Multiple-strikes may be repeated to achieve a desired outcome.

In real-time, provider determines if circling-back for retreatments is prudent or not. This “prior planning approach” commonly eliminates most nagging lesions from regrowth (as a recurring threat). A process referred to as Krieger-Amulet-TaaS℠ (KAT) method. Reducing unforced errors, retreatments and costs.

[FIGS. 9-11] A review of the cross-sectional images, IMP-metered socket-keys are shown as “dark-teardrop” images (medium and large). Accompanying each IMP is a corresponding Halo-Corona-Gap. As previously described, Level-2 encompasses all IMP-metered socket-keys (aperture-funnels). Where funnels provide for temporary LN2-pooling (cold-fusion). Also in FIG. 9, notice how top-down arrow shows direction of Stage-2 procedure: LN2 (or cryoprobe/cryopen) effectuating a top-down “air-strike”.

A full-cycle of DCP-D triage is on display. Wand-base (8) is shown as a horizontal, cross-section (despite the fact that Wand is substantially % pipe, arcuate.

FIG. 7 Image shows “digital-twin Stadium 3D half-pipe” oriented, MIT/IMP matrix. Amenities for cryotherapy in double-portions. Through-hole rendering of corresponding socket-keys.

[FIG. 9] Notice how a “downward pointed arrow” originates at the LN2 source; where LN2 flows down per gravity. Aimed at a 90° kill-shot angle of attack. Distal and proximal portions of Wand-base tilt, XYZ-axes, moon walk and 3D roll. Empowering user for quick, distal-to-proximal, rapid-fire holographic AI/AR triage, where gain of function attack vectors are imposed in a productive, friendly-fire, gain of function context. Life-saving, life affirming holistic-triage. Positive and repeatable. Purpose-built, fully operational; off-grid and minimalist.

In [FIGS. 10-11] we re-examine ADA/AMA recognized spray-pattern paradigms. Proper placement achieves full penetration. Forming an ice-field around a lesion (or lesion-cluster). Creating-cell destruction and necrosis to feeder blood-stalk supply. Also note where [FIGS. 10-11] infers: an LN2, cryo-gas spray (or probe, etc.) is directly applied/administered; shooting-through Wand base via a Level-2 IMP socket-key, creating cell-death necrosis.

Tactical, purpose-built, patient-centric; each Halo-Corona-Gap “holds the cryogen charge in-place.” On-point, with the lesion encased in a socket, able to maintain this Close-Quarter-Hold (CQH) is necessary to effectuate a 100% deep-dive hole-shot. Whether or not the particular lesion requires to be re-touched.

Prolonged stewing of LN2 produces superior end-results, allowing continued deep-dive penetration for 100% ablation. Causing fewer patient return visits, higher patient-triage satisfaction; and favorable provider reporting/scrutiny at social-media outlets.

An ICE-Offensive (hard-freeze) is in-play. Decisive-blow to morphologies. Concentrating, intensifying and multiplying the cryogenic-effect and destructive necrosis cycle is the purpose of Level-2 MIT band. Integration and pin-point accuracy. DPC-D high definition (HD) triage results. Spatially acclimated, this level of accuracy, marks a “clinical check-mate.” A mission accomplished moment. Accuracy always requires a caring, studied, observant, and steady-hand. As an aside, AI/AR 3D engineering, virtually eliminates “interstitial-gaps” between IMP-key and lesion. “No leaks equals no skip-shot. Quality triage means no malpractice litigation. No patient complaints or incriminating open-mike moments by disgruntled patients in social-media.” Tidy and clean triage equals patient equity. Creating traceability, accountability, with lower E/M “provenance-costs” to insurers. Serving as a “lesion-cleansing eradication campaign.”

[NOTE: During Stage-2 of triage, LN2 will “briefly freeze-stick” to the IMP halo-corona-gap. In the pocket (the drop-zone). Unique and specialized, tiered halos flash-freeze lesion-to-wand. Creating a high-definition, sheltered-in place, hole-shot execution. Should a provider have doubts of best practices; prudence demands seeking-out: professional-care, TS-DG/ISR, and clinical wisdom/experience BEFORE attempting triage procedures.

As a whole, all illustrations make the case: Stage-1 and Stage-2 must be performed accurately a.) According to ADA/AMA recommended clinical guidelines; and b.) In concert with Topside Stakeholder-Draft Guidance (TS-DG) Web link (provided on DCP-D's topside cover-label. Healthcare stewardship without spilling a drop (or breaking a sweat) where the only things dying are nasty lesions (and archaic bad-habits). Providing stakeholders with quick-draw, time-to-value, Key Performance Indicators (KPI's). With many fronts in this war, DCP-D seeks to “right the ship.”

Metered-Cryogen-Regulated+LN2 Spill-Shield CAPA Compliance

Doubling as a MIT platform, and a large reservoir/shield (11a, 11b). In an extreme emergency, if LN2 is accidentally spilled onto the Wand or patient during Stage-2; the cupping, arcuate, sled-shape of DCP-D Wand-base (8) serves “as an emergency elongated-capture-wand and gantry flick-ejector.” Protecting all parties; like a “burly baseball-catcher, guarding home-plate.”

Timed-Spot Freeze-Ejector-Port Technique+Jules-Thompson Method

ADA endorsed, DCP-D was designed around a co-witness surgical tourniquet. This accounts for: an optimum, metered dose of an LN2 biologic, applied to a specific lesion phenotype/size; administered through a pre-selected Matrix Metered Socket Protocol-key (aperture compensator). Direct, cross-threaded, auto-lock (ShureFit) preparation for a strategic, tactical; clean-biologic operation. Prepositioned, a cryogen is ADA-applied directly over-top; creating an ice-field. Properly done, this method will fully necrate the blood-stalk life-line; per AI Detail Enhancement (AIDE); limiting most re-visit/retreatments. Having a deep-bench, the AI/AR “mask-mandate of DCP-D” achieves FDA/CDRH regulations, and provider remittances (CMS.gov).

To some degree, each tourniquet-capture and choke-hold method results in the same standard of care. Personalized, 1-to-1 equity-inclusion-healthcare model. Freeze-exposure time frames are adjusted according to: skin-thickness, vascularity, tissue-phenotype, size, lesion morbidity, skin-folds, histology, etc. Maximum control, with minimum separation. Ablation with a proportionate, metered-pace without collateral damage. Lesion masked-mandate and border-wall paradigm for cryoablation, cryotreatment, cryosurgery, necrosis.

### VII. SUMMARY OVERVIEW

As a long-time triage out-patient, inventor is merely channeling his own (as well as other) frustrated, injured and marginalized patients. And so, inventor has painstakingly attempted to carefully delineate exactly how DCP-D functions as an Epidermal Lesion Removal Triage System. DCP-D measures a substantial length, width and thickness (per divisional/derivative and variant best practices). A disruptive, Med Tech device. Novel. Unobvious. No scarring. No excuses. No social-media open-mic moments. Ethical. Convenient pocket custodian on-a-stick. Human-Pocket Sherpa Form Factor (H-PSFF); performance triage achieving necrosis via Pressurized-Kinetic-Forces. Rx indications of Use.

A cache of resourceful amenities. A forward-deployed, mobile family medicine tool. Totally Off-the-Grid (OTG) having balance, ballast, a unique 3D XYZ axes. Broad-shoulders with a backbone. Courting and culling most skin lesions, on-demand. Full stop.

### VIII. Q&A CONSTITUTION OF PRESENT INVENTION

1. “Is there use of a constituent parts (per 21 CFR 4.2(e))? No.
2. Is there use of a combination part/biological agent? Yes.
3. List combination agents: LN2, a cryofreeze gas; cryoprobe.
4. Will an FDA application be filed? If so, where? Yes. CDRH.
5. If clinical studies have been submitted, is the submitter the subject of the Application Integrity Policy (AIP)? No, N/A.
6. Is device cGMP exempt? No.
7. Does device require the application of DC or AC electrical, or mechanical energy to operate/perform cryodestruction? No.
8. Does the device create or use radiological means, vibrate, use of ozone, flashing LEDs; asserting subjective or burdensome claims to restore a patient to a more youthful appearance; or use of topical acids, chemo-peels, IVD-needles, Botox? No.
9. In substance, is ADV-01 a medical primary-device tool, prescribed and used by licensed healthcare professionals (Licensed Medical Intermediaries) for daily clinic-care? Yes.
10. Was a Regulatory Pathway Assessment (RPA) completed? Yes
11. Recognized by U.S., ERA, MDR and CE regulatory bodies? Yes.
12. Was the device developed according to FDA eQMS? Yes.
13. Was the device peer reviewed by: FDA/CDRH; a CRO (Contract Research Organization) and Medical Research Org. (MRO)? Yes.
14. Is device CPT-coded ready (www.CMS.gov) for Medicare and Medicaid provider reimbursements? Yes.
15. Is DCP-D manufactured at an FDA cGMP licensed plant? Yes.
16. How is DCP-D produced? Injection-molding; thermo-molding/stamping; laser-sintering. Including .SLA and/or advanced 3D Imaging, via: forged, CT-scanned, hydro-formed, 3D CAD-CAM, 3MF, .STL, OBJ, FDM, PolyJet, cold-fusion; advanced Next-Gen 4D, gas-purged Zero-Gravity, magnetic-displacement, Nano-scale AI/AR process; Additive/Subtractive manufacturing; use of post-production shark-skin or similar over-layering.
17. What materials are used to manufacture DCP-D? Materials originating out of (but similar to): Medical-grade resins, ABS, Acrylic, Nylon, Polyethylene, Polypropylene; TPE's (Thermoplastic Elastomers); adding-in: blowing agents, biocide agents; including more extreme anticoagulant additives. Additional undisclosed agents, colorants, magnetic MRI isotope agents, medical grade silicon molding, over-molding; custom rubber formulations. Advanced CT scanned techniques that match and/or exceeds year 2022 Six Sigma (or equivalent). Nano-scale gRNA-tRNA array of CRISPR/CAS9 editing; polyamide-based, glass fiber reinforced, carbon, graphene; EPA recyclable; post-consumer recycled (high to low density), PCR resins and similar (PET to rPET) containing thermo-insulating properties. Further including current medical device regulatory standards found in: EU, FDA China (CFDA), Order 739, Hong Kong, Asia-Minor. Including: India, Indonesia, Japan, Korea, Indonesia, Philippines, Singapore, Taiwan, Thailand and United States.
18. Is DCP-D a regulatory standard compliant device? ISO Standards for Medical Devices: ISO 13485-2016 eQMS, ISO 14971:2019, ISO 9001:2015, ISO 62304, ISO 10993, ISO 15223, ISO 11135:2014, ISO 11137, ISO 11607, TS 16949, ISO 14000-ready (plus current European Harmonized standards)? Yes.
19. Is DCP-D an “Active or Non-Active” device? Non-active.
20. Does DCP-D have and/or use a color-in-molding? Yes.
21. Is DCP-D an IVD (or otherwise) implanted device? No.
22. Is DCP-D an FDA (CDRH) regulated device? Yes.
23. Is DCP-D Pandemic Deployment Compliant (PDC)? Yes.
24. Is DCP-D device, 3rd Party Review dependent? No.
25. Does DCP-D include a digitally-encoded filter matrix? Yes.
26. Is device difficult or easy to use? Designed for manufacturing and use, DCP-D is “E20-11” (Easy to Operate-11).
27. Will DCP-D use a label? Yes, per ISO 15223-1:2021.
28. Is DCP-D a HIPPA & HITECH compliant-ready tool? Yes.
29. What is meant by “Zero-G?” Weight-focused, lite-weighting effects glide-path and dexterity (Backbone-SRS). Integrated Under-Body Edge-Glide-Rails allow support-ballast precision.
30. What is meant by ‘Stadium Half-Pipe Redundancy?” During triage, user has identical mirrored IMP-keys on both sides of Wand-centerline. Quick acquisition sockets in triage.
31. Are there multiple (derivative) models of DCP-D that are not delineated, illustrated or examined herein? Yes.
32. Battlefield triage readiness; Is DCP-D substantially modeled according to Mil Spec-grade (MIL STD 810G-516.6)? Yes.
33. Is DCP-D a bona fide Mobile-Directed-Energy Asset (MDEA), and a STEAM qualified tool for treating many common skin-lesions including: basal cell, squamous cell, pre and post cancerous lesions, morphologies and epithelial growths? Yes.
34. Is there any chance of a misfire, jam, product failure? No.
35. Is DCP-D made in Russia or China? No. Made in the USA!
36. Is ADV-01 (Beat Mode of DCP-D) an Essential Services tool for cancer treatment, Elective Procedures and TaaS? Yes.
37. Does DCP-D (ADV-01) provide Multifactor Authentication (MFA) with Bio-motion Recognition; containing auto-lock funnel undercuts to safely accommodate gimbal-pivots per triage? Yes.
38. Is DCP-D agnostic, along with L.G.B.T.Q.I.A+ friendly? Yes.
39. Does DCP-D require an annual NIST or fee-based maintenance schedule, or require a periodic firmware upgrade-patch? No.
40. Is DCP-D a Medical Tourism, MASH compliant device? Yes.
41. Does DCP-D contain AI Detail Enhancement (AIDE) Mode? Yes
42. No assembly required; Is device turn-key ready? Yes.
43. Do device materials reveal environmental stewardship of wildlife for a sustainable, recyclable new green-economy? Yes.
44. Is DCP-D an off-grid, clean-energy; step-forward Med Tech alternative to ‘high-dollar’ NIST-calibrated machinery? Yes.
45. Does DCP-D mandate rules of conduct of safety? Yes. AI/AR maximizes a ‘360° Masked-Mandate and Border-Wall Lock-down’.
46. Is DCP-D Informed-Consent [16 CPR § 1028.16] ready? Yes.
47. Is DCP-D a Single Use Device (SUD); or does it contain L/T Total Product Life Cycle, having multi-use shelf-life? Both.
48. Does DCP-D ‘check the boxes & sticks-the-landing’ as a reliable, Med Tech based, family and emergency Rx tool? Yes.
49. Right out of the box, Is DCP-D a binary, user-interface, Ui/Ux surgical prosthesis; triage-ready and able for cryosurgery on patients without a need for: NIST calibration, set-up, registration, AC/DC charging or two physicians?” Yes.
50. Is device cordless, wireless, non-electrical; considered a 100% pneumatic-biologic, mobile-triage Ui/Ux experience? Yes.
51. Is DCP-D antimicrobial? Antimicrobial blending of Ionic Silver (and/or multiple custom compound formulations); plus healthcare and food-grade recognized additives (as needed) to combine singularly/severally to inhibit a degree of healthcare colonialization, Complicated SSTIs (cSSTIs) infections? Yes.
52. Is DCP-D considered a EUA Covid-19 readiness device; able to be sanitized safely; treat multiple-patients, preventing cross-contamination or risk of infection?” Yes (see above).
53. Does Spec-Ops-7 (UHD-7) serve to ‘depopulate-clusters’ of lesions; while forming an allied-alliance with insurers, based on shared-values, actionable intelligence and cryogen leakage-prevention? Yes. Is triage performed wirelessly? Yes.
54. Does DCP-D allow studied stakeholders to reach accurate assessments, promoting repeatable humanitarian relief? Yes.” Following a review of DCP-D, an examiner may well ascertain all essential checks and balances, safety by the numbers, etc. are in place. (Disclaimer: Proper UVA/UVB, autoclave, ethylene oxide sterilization ISO 11135:2014; ADA/AMA sanitation protocols must be followed (and compliant), before DCP-D monoframe is safe for reuse on subsequent patients; Total Product Life Cycle (TPLC).

DCP-D Requires 3-Stages for Cryotherapy Recuperation

once a lesion is found, the user has the opportunity (and Wand-based means) to measure, research phenotype; ascertain size and profile. Monitoring TS-DG assessments BEFORE proceeding with (or attempting) a “live cryosurgery/cryotherapy procedure.” Affording stakeholder every opportunity to gain medical advice in advance. Informed consent decisions, pre-screen surgery etc.; allowing all parties to invoke prudent medical pre-evaluations.

STAGE-1: Pre-Ablation; Shelter-in-Place/Survey Protocol. A targeted-acquisition (hide-and-seek) of a lesion/morphology is structured; clinical protocol (per ADA/AMA cryodestruction methodology). Lesion Screening-Identification (LS-ID). Where stakeholder (MD, Rx physician, NPP, healthcare provider or DIY home-user) squeezes Wand down-over-against a specific lesion (or cluster of lesions) inside the Cryogenic Landing-Zone (CL-Z). Forming an encoded “suppressor filter seal as a second-skin” interface between lesion and device (AKA pre-ablation staging).

STAGE-2: Gain-of-function; asymmetrical necration. Interface Ablation Sequence (IAS) begins. IAS is the actual application of a cryogen. Liquid Nitrogen Q-tip suave is preferred. In one example, when Liquid Nitrogen (LN2) is used with a lengthy Q-tip or similar applicator); the cotton swab tip is immediately pressed against a lesion. At −319° F., LN2 is tricky and may pose a serious and/or dangerous risk if mishandled or spilled. Expanding LN2 evaporates immediately at room temperature (from a vented Dewar or properly modified stainless-steel thermos).

During cryodestruction the user: co-witnesses, monitors and proceeds with the interaction, making real-time calculations and adjustments; determining if circling-back or not to kill blood-stalk. Forward deployed, asymmetric-guerilla-warfare on single (or multiple) lesion-cluster is de rigueur. Battle-ready per tactical/skillful use of Spec-Ops-7/I-OPS. CMS.gov reimbursement compliant. Platform-ready to triage scores of lesions per visit.

STAGE-3: Marks a natural, regenerative-healing process performed by the body (for most normal humans). A de rigueur cycle that leads harmoniously toward a copacetic, holistic resting-state.

IMP Metered Socket Protocol-Keys (Aperture Compensators) Perform Four (4) Critical Functions:

1. “Over-flow capture of an LN2;” sustaining the formidable cryogenic charge “just a little bit longer” as it's working its “thermobaric bunker-busting magic” over the lesion.

2. Providing a “safety-net” (safety-gantry function); a Wand length reservoir of cryogen allows for sustained procurement of any small and errant “over application” of LN2 (should that accidentally occur) during Stage-2. Correctly administered, a structured/predictable snow-cone effect occurs.

3. Lesion will temporarily freeze-stick (clinical cold-fusion) to the IMP socket-key aperture; holding it in place (tourniquet-effect) under the scorching, penetrating, punishing pressurized forces of a cryogen charge. During which time the user holds-steady, making observations. Attentive and prepared to pivot or abort triage fast! Acting holistically on behalf of the patient.

4. A “cryo-firewall stand-in.” A patient-safety mechanical barrier. A careful “bubble-defense isolate” surrounds lesion.

Protecting, shielding adjoining unaffected tissues, from skip-shot collateral damage. DCP-D may also be used along-side: homeopathic salves, topical salicylic acid, cauterization; topical brush-on chemo skin-peeling (other ADA approved means). In substance, device material(s) were “biocompatibility tested” to ISO 10993-1:2009; medical-grade blending's enabling: reflex, 3D miniaturization, IMP-mosaic socket-keys, under-cuts, surfing and crawling, coloration and repeated use. Metered delivery systems. Tight, circumferential borders with no premature easing of restrictions at the margins. Entire 360° circumferential edging of Wand is an open-halo for triage. A Rapid Defense-Rapid Offense Procedures™ (RD-ROP); safe as a Pandemic Deployment Compliant℠ device and family medicine tool. Mission specific sorties, triage and special operations for disaster relief. A panacea addressing safety, security assistance and security guarantees for stakeholders and patients. De-risking the stress. Damage-control from the brutal mechanisms of prior art follies.

### IX. STATEMENT OF INVENTION

A one-handed, asymmetric, strike-force on-a-stick. A physician extender for Licensed Medical Intermediaries (LMIs), NPPs and DIY home-converts. Aspirational. Setting a new standard-of-care in triage cryosurgery. It is advised that stakeholders using DCP-D, first seek medical training, certification(s) from an attending physician and Stakeholder-Draft Guidance (S-DG) Web link PRIOR to administering cryotreatment/cryodestruction.

[NOTE: Inventor/sponsor advises that users and caregivers review and follow all training and regulatory standards/certifications. I-Ops Munition IMP socket-keys (15), and the proper use of the Spec-Ops-7/I-OPS; pin-point triage by the numbers. Allowing for fidelity, discretion as a Level-2, pocket-Sherpa, form-factor. Single-action, center-fired, aperture-suppressor. A full suite as a clinical, plug-and-play, physician extender. Timed-spot operation using socket-keys per “freezer-locking breach.” One-and-done triage neutralizing epithelial lesion outgrowths. DCP-D (ADV-01) is considered an “Equal-Opportunity, Assistive Copilot during a cold-war renaissance of HD patientcare.” Intervention using Interdiction Consumer Empowerment (ICE), avoiding injury, malpractice and blood-letting conflicts. Bringing to bear novel, bespoke, gravitational and pressurized kinetic-forces; without use of electricity, constituent parts, or 3rd party calibration.

### X. SUMMARY

Asymmetrical, hand-held. DCP-D is an all-in-one, Tissue Defense System 101. Pared with a 3d party biologic, effective as a: “bilateral, Andro-Synchronous, Level-2, Skeletal-Girder Reflex Exoframe tool.” Performing triage in high-definition (HD). Surveying and performing epithelial examinations copilot (TS-DG) training for stakeholders. Deliverables are indicated to improve doctor performance review by eliminating misguided injuries, shaky-hand foibles; skin-burning misadventures. Safe and sobering outpatient triage for all patients, both young and old.

[DISCLAIMER: DCP-D (ADV-01) aspires to support and reinforce a go-to leadership role for in and out of network clinics. Eliminating the obstacles and inefficiencies of embedded predicates (prior art). DCP-D meets Federal Drug Administration (FDA) 21 CFR instrument for general use (Class-1 General Controls). Applicant-inventor claims and specification disclosures are intended to achieve: USPTO statutory enablement provision 35 USC S 112(1)/(a); post AIA. Filed as an NGO.

35 USC 112 (1)/(a): “The specification shall contain a written description of the Invention, and of the manner and process of making and using it, in such full, clear, and concise, and exact terms as to enable any person skilled in the art to which it pertains, or with which it is most nearly connected, to make and use the same, and shall set forth the Best Mode contemplated by the inventor or joint inventor of carrying out the invention.” DCP-D cryotherapy; TaaS methodology safely executes its duties in a unique, unambiguous manner. Literally floats upon a Backbone-SRS; guided by underside edge glide-rails allow for a counter-steer functionality. A ground-up performance matrix able to pivot/maneuver unflinchingly. Life-cycle reusable under: UV/IR clinical autoclave; including advanced ETO, Gamma, E-beam, X-ray, NO2 sanitation methods (and the like)].

Moreover, all critiques of prior art devices was based-on objective, printed matters of fact. Spotlighting how aerosolized predicates have “weaponized” chemical-biological agent(s) as unsustainable, stymied and onerous. Many pedicates promote the launch of “fire and forget” (FAF) lethal doses of cryogens. USPTO allows truth-telling without: surmising, malicious disinformation or gas lighting critiques. Welcoming dissenting points-of-view about the pathology of embedded devices in objective, dispassionate terms. As a one size fits all stand-in, many challenger devices exhibit strategic ambiguity. Painfully flawed. Dysfunctional. Evasive accountability. Yet mentioned without trade-libel, false claims or disrespect. Each inventor exercises their own brand of ill-equipped, incompetent, regressive-archaic measures. Inflicting long term harm as their Best Mode devices. But for a side-by-side comparison; 3^{rd} party observers will independently decide if DCP-D/ADV-01 prosthetic, can safely out-perform (out-gun) predicate devices, without capitulating to draconian-standards or nixed triage results.

ADV-01 is a skills-based, plug-and-play, TaaS Med Tech model. Utilizing unique nomenclature. Timed-spot, Freeze-Locked-Breach (FLB) methods, etc. Tightening the triage-circle around unwanted tissue(s). Advanced metered features depopulate singular and lesion clusters; attacking lesion(s) at feeder blood-stalk, per Core Customization triage. Reusable, sustainable, recyclable material (s). Advanced composites; organic/inorganic, thermosets, glass fiber, Additive Manufacturing Compression Molding (AMCM), and Subtractive Manufacturing (SM) methods of development. Top-side label (TS-DG) tutoring via Intel-Surveillance Recon (I-SR). Personal Mobility Device (PMD); mobile lesion processing-unit. Discrete monoframe. Back-fill solution for cryotherapy. A reset for outpatient triage eliminating “off-script, Hail Mary guess-work.” No-fault leadership for: humanitarian crises, disaster relief, MASH; family and emergency medicine. Vociferous advocate for accountability, serving long-suffering patients ISO a safe, authentic, even-handed surgical experience. A practical, handy, reliable, primary-device for LMI's, NPP's, conscripts, home healthcare and arm-chair practitioners. Mercy-Aid Med Tech. Patient-centric translator, producing 3-Stages of Blitz-Krieg epidermal restoration. Industry-wide lesion cleansing campaign for patient-honorees. “As provider sows, a patient will reap” perspective. Triage with CAPA security guarantees built-in. Democratized Humanitarian-Aid on a Stick (HAS). Declassified./RK

### BEST MODE

(Described in Detail Per Specification DCP-D/ADV-01)

### MODE OF INTERVENTION

N/A

### INDUSTRIAL APPLICABILITY

N/A;

### SEQUENCE LISTING

N/A

I claim:

1. A 3D Halo-Deck Wand Level-2 Exoframe chassis (8) (which comprises);
a bilateral, symmetrical, arcuate, open, HD Stadium 3D Surround-Vision Triage-scape Arena Architecture; a flexible half-pipe platform; a base also containing a Morphological Index Template (MIT), a first side and a second side, both sides having Index Morph Patterns (IMPS) with a plurality of mechanical, menu-engineered, biomimicry, morphological key-apertures on the first and second side of a central center-line; having a native-anatomic vector-grid pattern, 3D positioned throughout the Wand-base, and shaped substantially as a Skeletal-Girder Reflex Exoframe; a jeweled-mosaic, half-pipe elongated bowl; a Topside Distal and Proximal Tiered-Halo Deck-Band-Corona, forms said Level-2 upper-deck, and is substantially integral throughout the Wand-base Exoframe chassis nomenclature; the terrace is concurrent among the vector patterns; creating a raised Augmented Reality (AR) functional amenity to the terrace step layer; oriented tangent upon, and circumferentially to said 3D Halo-Deck Wand-base (8) as a breathable, slide-polymer monoframe; having a Under-Body nomenclature accompanying said Skeletal-Girder, Wand-base Exoframe chassis; a la carte.

2. Halo-Deck Skeletal-Girder Reflex Sxoframe platform of claim 1 further comprising:
a topside of the Level-2 Exoframe chassis, shaped substantially as a 3D, semi-rigid, open, “stadium half-pipe oriented” semi-solid Skeletal-Girder Reflex Exoframe; a 360° Technology as a Service (TaaS) Rx medical tool platform; an offensive and defensive asymmetrical cache-ensemble device with the key-aperture mosaic matrix native patterns comprised of the circumferential Morphology Index Template (MIT); wherein a subset incorporates a plurality of holographic otoscope speculum as an Indexed Morph Pattern (IMP); wherein IMPs containing said metered-socket protocol-keys matrix includes undercut-funnels formed throughout Reflex Exoframe chassis; Multichannel Direct-Input:1 (MDI:1) decodes Analog lesion-Inputs, against Digital IMP-encoded Outputs (DAI-DIO); constituting Distal/Proximal apertures forming “a specialized menagerie of numbered, metered IMP Cluster-keys” as Artificial Intelligence (AI); augmented Reality (AR); wherein said IMP mosaic fixtures produce a Tourniquet Capture Isolation (TCI); Close-Quarter Hold (CQH) under special conditions; able to narrow and widen footprint corridor, closing-in as the circle tightens around unwanted tissues; use of an asymmetrical, feature-rich matrix-mosaic-mosaic Level-2 pattern throughout Wand chassis; producing high-definition (HD) surgical procedures per American Academy of Dermatology (AAD), American Dermatological Association (ADA);
whereby said Level-2 is a confederation united by a circumferential MIT raised band; emergency Use Authorization (EUA), Personal Protective Equipment (PPE) compliant-ready for cryotreatment SOPs in a novel (new) coronavirus (nCoV); facilitating a Stage-1, Androgenic-Epithelial-Surveying (AES), and a Stage-2 cryotherapy triage sequence of events; incorporating a Core Sync, Spec-Ops-7 (UHD-7), triage-by-the-numbers, per an I-OPS (Input-Outputs Per Second). A novel approach for cryodestruction during a break-out pandemic; allowing device to perform as a Pandemic Consumer-Deployment-Compliant℠ method, wherein said specialized IMP protocol-keys consist of individual, open-cone aperture funnels, having varying sizes of said native biomimicing shapes; apertures that (in-substance) will direct, meter, regulate, govern a flow of LN2 (or guide any qualified cryomeans); a digitally encoded-filter membrane that shape-shifts a lesion thru each decoded IMP socket-key filter; where the shape-shifting IMP-keys are located throughout said distal and proximal Level-2 halo-deck of elongated, half-pipe, linear Arcuate Bowl Architecture (28a-28b) of said Skeletal-Girder Reflex Exoframe chassis providing Core Customization in triage vis-à-vis digital-twin bandwidth;
tasked with Rapid Triage Elements (RTEs); Lesion Screening-Identification (LS-ID) and interrogation, plus a momentary-freeze-interrogation (per a leveraged-suspension dynamic) of proprietary Tourniquet-Capture-Isolation (TCI) over common ADA recognized, epithelial anomalies etc.; common lesion growths occurring on the outer visible epithelial surface of the epidermis; on people in otherwise good-health, without secondary, exotic and/or extreme radiological preconditions;
said Morphological Index Template (MIT) contain said subset of IMP protocol-keys (IMPs); wherein, Indexed Morph Patterns represent a plethora of anatomic biomimicry of flow-metered, cryogen applications; through-hole, protocol-key apertures, throughout said 3D Exoframe Level-2 terrace; producing an Open-Source-Technology (OST); unlocked, apertures facilitate a full 360° Panoramic-Site-Picture (PSP) via Panoramic, balanced and HD Stadium Surround-Vision Triage-scape Arena Architecture; said IMPs produce a unique “Braille-Effect” connectivity, acting as Braille picture-puzzle-pieces; 360° maneuvers are possible via said half-pipe architecture of said HD Stadium Surround-Vison Triage-scape Arena; Backbone-SRS, said Underbody Glide-Rails;
a bioengineered work-around that is unique and unobvious for HD performance and “no-fault” cryotherapy procedures;
IMP Aperture protocol-keys are overt interrogation-tools used during said Stage-1 Androgenic-Epithelial-Surveying (AES);
wherein said Stage-1 surveying and clinical assessment to identify suspect surface living tissue(s) lends to the Stage-2 ablation to cull, necrate and destroy unwanted tissues per: auto-lock, Self-Indexing-Compensators (SIC) built into a majority of IMP-protocol aperture keys; producing auto-lock Close-Quarter-Tourniquet (CQT) that seizes-lesions; reinforcing command and control over lesions taking casualties; including small-scale, top-down surgical air-strikes, to full-scale customized patientcare across an entire range of patient needs;
necration/cryodestruction process per said jeweled-mosaic aperture key is actuated by secondary, two-stage, stakeholder activities; wherein said IMP-halo corona feature temporarily creates a reservoir; storing a predetermined, metered dosage of a biologic cryogen (or qualifying cryoprobe, cryopen-type) or similar cryo-inducing methods, laser-scalpel methodology services or devices; occurring in an ambient operating room-temperature (ORT) or simulated, Weightless ORT environments under substantially evaporative effects; said Halo is a novel High-Altitude, Low-Opening (HI-LO) corona around a limited number of said IMPs, apertures, orifices, mosaic-keys, AI/AR or holographic otoscope means, measures, intonations or the like;
cold-fusion, flash-frozen and stored substantially inside or tangent, adjacent to said pre-selected IMP protocol-key aperture matrix; adding said combination biologic refrigerant to create said flash-freeze and/or snow-cone effect over a tourniquet-capture, sheltered-in-place tissue; courting and culling said lesions; precipitating tissue-morphology into a denatured and lifeless state of cryo-necrosis; ceasing growth (as in life), spores, procreation or the like targeted (or randomized) tissue by killing-off its critical life-line blood-stalk supply in real-time or through a structure/unstructured delayed destruction deep-freeze, or irradiation process singularly or severally; including an ADA, AAC, or AMA recognized Jules-Thompson Methodology of cryotherapy/cryodestruction;
wherein said Exoframe platform consists of a Under-Body Distal Matrix (30a), and a Proximal Matrix (30b); whereas the matrix is bisected by a central/centered Under-Body Backbone-SRS (26); designed as a co-pilot for discrete XYZ-axes: tilt, pitch, yaw, roll; cross-thread, moon-walk, gimbal, forward and Switch-360° Reverse Switch (clock-wise/counter-clockwise rotations);
gimbaled-dexterity interfaces upon user's fingertips to maneuver device, effecting triage fidelity; ADV-01/DCP-D incorporates forming, molding and similar shaping processes; produced per chemical impermeable materials and the like, but similar in substance to resins, durable Nano-tech coatings, micromachining notwithstanding; the floating Backbone-SRS (produces a ballast-effect); providing for a gliding Zero-G, micro gravity tool; effecting real-time user bump-feedback loop, transparency and accountability; Confident, smooth-gliding; a safe technical execution with unblemished results; said Level-2 Exoframe-Wand chassis performs across a natural-range of: bumpy, bleached, smooth, curved, sweaty, scabbed, diseased scarred and/or hairy epithelial skin-folds; including tight, ruff, squeamish private-parts; employing Under-Body Edge-Glide-Rails for the XYZ-axes maneuvers; an interplay of complex factors, maintaining a track-position for efficiency and clinical protocols where needed; intervention using Interdiction Consumer Empowerment (ICE);
a depot of Just-in-Time (JIT) assets utilizing a patented Snag Reduction System [SRS] in concert with Backbone, preventing unintended, undesired snags, cuts or the like during Stage-1 Androgenic-Epithelial-Surveying (AES) pre-ablation checklist; Holographic Augmented Reality (HAR); hide-and-seek, in-situ prospecting using: compact, single-action, center-fired, optics-ready, aperture suppressor; a plug-and-play, timed-spot with Freeze-Locked-Breach (FLB); IMP-key ejector-ports carry an estimated 100:1 kill-ratio over lesions; bringing to bear bespoke gravitational, and Pressurized Kinetic-Forces (PKFs); discrete, skill-based cryotherapy, proctored by stakeholders;
forging a discrete, slide-polymer, sled-like (or similar) Skeletal-Girder Reflex Exoframe; providing for a semi-automatic, LN2-loaded and liquid fired, disruptive Med Tech tool, containing a subset depot of IMP jeweled-mosaic protocol keys; funnel-like apertures used for surveying, inspection, cryogen pooling; native IMP portal-keys acting as a scalable, vector-arsenal-asset-cache, transformational, cryosurgical tool-set;
advanced technologies; capabilities of the AI Detail Enhancement Mode (AIDE) utilizing said Under-Body Edge Glide-Rails respectively; wherein Edge Glide-Rails counter-steer hardware included; via said AI/AR maximizes said Wand per a 360° Masked-Mandate Rule for improved surgical and patient care;
intended for improved Inputs, Outputs, Indications and Validation(s) of Use; also intended to restore confidence and/or the public-trust; providing user with substantially a one-and-done CDRH, FDA compliant hybrid STEAM multi-tool;
human factor, medical carrier strike group form-factor, providing offensive ablation over targeted lesions; with a corresponding asymmetrical defensive-shield of protection surrounding virgin unaffected tissue; under AI/AR and assorted a la carte cache of said native biomimicry assets, consistent with or similar to Holographic Augmented Reality (HAR), RTE's;
inspiring, empowering a prolific, 1:1, 2:1, 3:1, etc.; end-to-end, 360° rotational, asymmetrical Pivot to accommodate a Patient; full-on user-interface functionality; supporting or providing an up-stream number of clinical phenotypical results or benefits; a non-contentious Esprit de Corps post-operative treatment; and/or post-surgery results as a result to partial, timed, delayed or completed/executed cryodestruction methods.

3. Where improvements to Exoframe (8) in claim 2 consisting of; a bisecting Mid-Ridge Groove (32) for tactical-grip and positive hand-grip fidelity with real-time feedback while culling targeted tissue(s); a specialized Florentine-styled elongated aperture is provided as a work-around lesion capture protocol-key of said Level-2 Skeletal-Girder Reflex Exoframe chassis; where existing IMPs is, are or may be surgically insufficient;
a depot of said native proprietary hybrid IMP-key aperture funnels having top-to-bottom, 3D under-cut termination ending points; plus the Florentine-styled jeweled ensemble-key, allowing for a tactical, Self-Indexing Compensator (SIC) protocol means for a deliberate, targeted, focused safe-shielding; tangential under off-set (or 90° direct) 3D-AR cryotreatment method using pitch, yaw, pivot and roll (cross-threading) articulated, proprietary Rapid Defense-Rapid Offense Procedures™ (RD-ROP); for use prior during or post Stage-1, and Stage-2 cryotherapy; considered graceful and sinuous, IMP effectuate protocol-keys gimbal per digital-encoded, auto-lock spatial acclimation; idiosyncratic, in-focus IMP's where lesions are singular or in clusters; providing said cryogen-means through said I-OPS, Spec-Ops-7 munitions to transform, catalyze, effectuate fleshy lesions inside, along-side or outside of a structured edge, aperture, or the like; having a dedicated tail section provides “slide-in and crimp sockets” where a 3d party biologic creates said denatured state to tissue, lesion, morphology, oncological structures or the like; cascading in sluffing-off and regeneration per natural healing, and/or under simulated, artificial assisted healing/holistic means;
achieving cryosurgical necrosis under proscribed (multi-vector) gravitational, plus Pressurized-Kinetic Forces (PKFs) as FDA recognized Med Tech Indications of Use;
said Skeletal-Girder Reflex Exoframe Wand incorporates tangible, ergonomic, svelte lab-coat, pocket-sized Human Form Factor (HFF) presence, a non-active, no-moving parts (with no constituent parts or sub-assemblies), using said over-arching iconic half-pipe orientation;
providing FDA 21 CFR Parts 812-892 (polychromatic labeling and tutelage); where said improvements of AI and AR provisions further include: FDA Section 201; 502(f) (1) and (2) Fair Packaging and Labeling Act (FPLA); Exoframe is regulated as a: non-IVD, non-active (non-electrified); FDA/CDRH Class-1 medical device authorized for the application of a 3^{rd}-party refrigerant (CDRH combination-biologic) during said Stage-2 cryotreatment; based upon the Jules-Thompson Method;
Under-Body Snag-Reduction-System (SRS) using a raised, semi-rigid Backbone, producing a full-suite of Zero-G micro-gravity XYZ advantages, per gimbal gyration-dynamics, in a stadium, half-pipe oriented Digital-Twin; a wide-band aspect ratio in an Analog-to-Digital filter format; where apertures also control throughput biologic consistency, a lesion-to-workflow smack-down; incorporating a plurality of Under-Body Edge-Glide-Rails; a pro-consumer, multi-discipline medical triage-filter device, cross-industrial performance operator administering precise Ui/Ux integration, to obtain a multi-vector validation by: FDA/CDRH/MACRA/CAPA/ISO/CE/EU/CFDA regulatory stewards as needed;
an asymmetrical all-in-one epidermal lesion removal prosthesis system, in asymmetric and double-proportions; Life-cycle reusable under UV/IR clinical autoclave and advanced: ETO, Gamma, E-beam, X-ray, NO2, organic/inert sanitation methods; said Rapid Defense-Rapid Offense Procedures' include; a dual-offensive and defensive risk reduction safeguard; regulated by U.S. FDA (www.fda.gov); Class-1 medical device (hospital use); FDA product code under 21 CFR Parts 862-892; Wherein, ADV-01 also incorporates derivative-divisional models (in the DCP-D ecosystem) ranging in size: 48 mm L×20 mm W×4.23 mm-88.9 mm T; DCP-D/ADV-01 contains variations plus/minus within that range. Amenable for a comprehensive ecosystem of device. Including said Level-2 AI/AR masked-mandate and ICE border-wall functionality; Distal-Head, Broad-Shoulder Wing-Segment and Hip-Segments have open-halo, chamfered, beveled, rounded-edges for ease of insertion into tight, hard-to-reach tissue areas;
FDA Class-1 said Level-2 Skeletal-Girder Reflex Exoframe chassis with Stage-1 cache; Lesion Harvesting Strategy of claims 1 and 2, further consisting of a Stage-2 Rapid Defense-Rapid Offense Procedure (RD-ROP); use of 3^{rd} party biologic(s) plus;
3D Halo-Deck Wand-base Exoframe perspective view [Top-Side]
3D Halo-Deck Wand-base Exoframe perspective [Under-Body]
Arcuate Bowl Architecture [anatomic arcuate ergonomics]
Level-2 Halo-Deck Distal-Banded-MIT-Corona [CQH portals]
Level-2 Halo-Deck Proximal-Banded-MIT-Corona [CQH portals]
Distal-Head [with Under-Body stabilizing edge-glide-rails]
Up-bent Arcuate Neck Segment
Spec-Ops-7 IMP Cluster Socket-keys [Metered IMPs]
Broad-Shoulder Wing Segment
Svelte Waist Segment
Hip-Wing Segment
Under-Body Edge-Glide-Rails
Ankle Segment
Tail-Bowl IMP Halos [proprietary protocol-keys]
Proximal-Tail Up-bent Segment
Tail-Edge Open Halos
Under-Body Backbone-SRS [Zero-G, micro-gravity; XYZ-axes]
Distal Linear Arcuate-Bowl Architecture
Proximal Linear Arcuate-Bowl Architecture
Florentine Styled Elongated Aperture [proprietary IMP]
Florentine Styled Elongated Aperture [proprietary IMP]
Under-Body Distal Matrix
Under-Body Proximal Matrix
Bisecting Mid-Ridge Groove
MIT/IMP Distal Matrix [metered-socket protocol-keys]
MIT/IMP Proximal Matrix [metered-socket protocol-keys]
Skeletal-lattice, mosaic half-pipe; a holographic, cutaneous specula otoscope; STEAM-designed Mercy-Aid;
Just-in-Time for Licensed Medical Intermediaries (B2B); and B2C DIY (OTC); Dialed-In, Squeeze-Roll Tunicate Capture, Tilt-Pitch-Gimbal-Pivot-Roll; Catch-Pitch-Ablate, Cross-Thread, Moon-Walk, Auto-Lock, and Switch-360° Reverse; a Lesion-Harvesting-Strategy (LHS) able to deliver a direct-hit and sustained necrosis per thermobaric bunker-busting effects; road-tested for all stakeholder segments; an easy-to-use exercise-skillset; DCP-D is a proprietary, Open-source asset for compatible markets, based upon Best-Mode (arête-folio ADV-01) Med Tech asset; IP68; IPX7, IPX8 and IPX9 compliant-ready arête folio; forged from laser engraving, injection-molding, thermo-molding, stamping, laser sintering, .SLA and/or advanced 3D processes via: hydro-form, 3D CAD-CAM, 3MF, .STL, OBJ, cold-fusion; advanced AI/AR, including Next-Gen 4D “intangible processes” per Weightless gas-purged Zero-Gravity, Computed Tomography (CT), magnetic-displacement, and AI/AR 2.0 processes (or the like); natural, simulated or artificial growth-means or anti-gravity, lattice, solid, artificial holographic, recycled, optical or sound-generated methods, Nano-sublimation means of construction; added-on post-production “shark-skin” or similar over-layering; polyamide-based, glass-fiber-reinforced; Exoframe platform; Paired with said modern, jeweled, bilateral, positive and negative biased RD-ROP; providing a plurality of Artificial Intelligence (AI), Augmented Reality (AR) inside a Block-Chain, NGO, Med Tech ecosystem Metaverse vis-A-vis ADV-01 HaMD model; facilitating a highly detailed hybrid-template for next-generation: strategic, tactical and operational protocols for providers of cryosurgical destruction;
health tech arsenal of holographic attributes, including a targeted Panoramic Site Picture (PSP); Co-Pilot 360° auto-lock; the Border-Wall 360° Masked-Mandate Rule; Spatially Acclimated, Anatomic (AN) handle of Wand-base is 1:1 physician extender (provisioning enhanced); ergo-designed for comfort to meld with the hand; able to survey and body-surf; gliding-across the skin; Stage-1 primed, JIT for pitch, yaw, gimbaled-roll with skilled 3D-control; DCP-D tool is removed from each lesion following Stage-2 ablation (per Immersive Extract Mode (IEM)); Arête-folio asset used in a cold-fusion medical procedure; E20-11 (Easy to Operate-11); prioritizing safety-efficiently and Technology as a Service (TaaS) in conjunction with Hardware as a Medical Device (HaMD) in said health Med Tech or similar life-sciences, MASH, medical-tourism or similar healthcare, patient industry-agenda;
presenting an iconic, elongated half-pipe Aperture-Wave-Design (AWD); in the form of said Skeletal-Girder Reflex Exoframe, pocket-sized form-factor chassis; said flexible, on-axes, 3D Exoframe Wand-base is in-situ; built upon a physician's multi-tool prosthesis; for purposes of outpatient 3D, high-performance cryotherapy and ICE (Interdiction Consumer Empowerment) as a unified gestalt, Lesion Harvesting Strategy (LHS);
a multi-disciplined, safety-based, STEAM-enabled, Gain-of-Function (GOF) discrete triage primary-device employing AI/AR novel features; based upon an array of proprietary, learned and bespoke ergonomic skillset (as all-in-one combination device);
non-active, non-moving, containing no sub-assemblies, nor constituent parts; Humanitarian Aid on-a Stick (HAS); First-responder, Industry Rescue-Tool (IRT); a depot of modular Intelligent-Novel Assets-On-Demand (IN-AOD); providing underside of Wand chassis with 3D X-Y-Z axes-attributes; Select IMPs contain variable point, multi-vector, hybrid star-ratio motifs;
as a secondary process, DCP-D is 3d party amenable per said biologic usage and dependent as primary mode of action (PMOA); enfranchising DCP-D as a bona fide FDA Combination-Device (consistent with FDA section 503(g) (1) regulations);
a medical triage platform approach using: gestalt, STEM/STEAM, Krieger-Amulet-TaaS™ method; and advanced healthcare 2.0 engineering principles; Divisional DCP-D (ADV-01) best mode variant uses AI/AR; representing a rudimentary Holographic Augmented Reality (HAR) platform within a wide-ranging divisional ecosystem for treatment upon: human, animal and “other” living (and post-mortem) organisms; a cultivated, asset-rich; first responder primary device for B2B and B2C stakeholders; a gate-keeper for CAPA-qualified cryotherapy;
providing actionable Stakeholder-Draft Guidance (S-DG) .pdf Web link for stakeholders; 3D Halo-Deck Wand is a binary user-interface; Ui/Ux surgical prosthesis; MIT is a design-canvas-matrix characterized by a Level-2, upper-deck, perimeter band that is thicker than a majority of said Skeletal-Girder Reflex Exoframe; notably, MIT contains a subset cache/ensemble of bucchero, IMP socket protocol keys; funnel-like apertures used for surveying, inspection, cryogen pooling;
Wand uses said existing native IMP portal-keys as a scalable, vector, asset-class-toolset; including entire, outer, circumferential edge of Wand that may serve as an unlocked open-Halo; conceived (in substance) from the STEAM paradigms; and comparable to proprietary (and open-source) 3D rendering tools;
a surgical technician-physician's tool useful as a functional work-around toolset; replete with full tactical gear on stand-by; considered an ensemble of anatomic cryotherapy assets in a single-unit (non-constituent part), combination-device; manufactured from substantially inert, surgical-grade materials originating out of (but similar to): FDA and or cGMP-compliant common-to-exotic material(s), and formed per chemical impermeable matter and/or similar to resins, plastic, metal, alloys, substrates, silicon, organic, inorganic, magnetic, radiated, irradiated; singular or multiple combined materials; UVA/B, electrified-fields, holographic-matter; Nano-layering; may be re-produced by titanium software (or similar) where materials are similar or a dissimilar in: nature, origin, means, composite and/or exotic sourced composites and/or method(s); formations sourced or engraved substrates or the like;
responsible and efficient; CAPA Safety through clinical checks and balances; Modular, integrated, DCP-D is intended as an Open-Source-Technology (OST) per bespoke Krieger-Amulet-TaaS™ method; AAD and ADA compliant “border-wall of intervention” against skip-shot botched-triage syndrome; DCP-D tool is pandemic and socially-distant compliant; gradient vectors of IMP sizes allowing a stakeholder to command-select an appropriate aperture vector (IMP-metered-socket protocol-keys protocol-key); where full-suite gradient 2 mm, 3 mm, 4 mm, 5 mm, 6 mm, 7 mm, 8 mm orifice IMP aperture Spec-Ops-7 encoded cluster-keys, comprising a 360° Lesion Masked-Mandate Rule; where full-suite Spec-Ops-7 gradient (IMP-subset factor of two) are Match-Grade tools for Interdiction-Consumer-Empowerment (ICE) protection, circumferentially/mechanically isolating lesions/morphologies etc. from adjoining/surrounding unaffected virgin tissue(s); mechanical capture; aperture-silo barricade-effect through extended-exposure inside a drop-zone of said Exoframe Wand-base (additive-subtractive manufacturing);
encoding tissue by gently facilitating an accurate 1:1, ID digital-encoded via the tourniquet-capture into an orifice or edge or similar means of said Wand-base Exoframe nomenclature;
providing a safety-net gate-keeper with said masked-mandate per mechanical means causing, creating a border-wall and isolated effect(s) per said IMP-vector arsenal, cache; circumferential ensemble; a disruptive “medical-tool with a spine” per said Backbone-SRS; left and right of center bead/spine, ADV-01 platform becomes a digital-twin; pairing the best IMPs of one-side, against the other; dual-ablation tools in-one; providing users with an HD Wide-band Aspect Ratio (HD WAR); combined with said Level-2, Spec-Ops-7 cluster-keys [numbered & metered IMPs in millimeters]; 3-Stage Lesion Mitigation Strategy: two (2) being provider proxy mechanical; whereby the third is nature's own skin-regeneration-healing cycle;
DCP-D made from Medical-grade resins, cGMP, ABS, Acrylic, Nylon, Polyethylene, Polypropylene, Thermoplastic Elastomers (TPEs); adding-in: blowing agents, biocide agents; including more extreme anticoagulant measures. Additional undisclosed agents, colorants, magnetic MRI isotope agents, medical grade silicon molding; extrusion; lattice polymer fiber resins; over-molding; Advanced Computed Tomography (CT scanned) techniques that match and/or exceeds year 2022 Six Sigma (or equivalent); jacketed, Nano-coatings; gRNA-tRNA array of CRISPR/CAS9 biologic editing or mRNA methods; polyamide-based, glass-fiber reinforced, carbon, graphene, Teflon®, titanium, gold, silver, stainless steel, amorphous material(s) and/or the like; EPA recyclable; post-consumer recycled (high to low density), post-production, renewables and recyclables' as Single Use Device (SUD); PCR resins and similar (PET to rPET); containing thermo-insulating properties; advanced composites; organic/inorganic, thermosets, glass fiber, Additive Manufacturing Compression Molding (AMCM) and Subtractive Manufacturing (SM) methods of development; sustainable, circular economic-thinking notwithstanding;
a monoframe, mobile-carrier strike-group wand; a quick-draw counter-measure using said Input-Output Operations Per Second (I-OPS); advantageous for stakeholders on the front-lines, in the theater of cryosurgical operations or battlefield war zone; in the weightlessness of space or under-sea (subsurface) triage; dialed-in of said full-suite Spec-Ops-7 gradients; a unique, non-obvious; crafted-blend of artisan-styled, ease of use Med Tech tooling; a pragmatic Blitzkrieg life-sciences initiative;
said cryodestruction treatment occurs per unique interface of: acquisition, intervention and interdiction triage; eliminating collateral-damage, pain and suffering; technology transfer of the cold-fusion methods; equity and respect ending the agony and loss of life due to preventable skin-cancer procedure(s);
entire 360° circumferential edging of Wand is an open-halo for triage; Stage-3 Blitz-Krieg rebirth initiative “as one sows, another will reap;” open-source Med Tech general hospital and primary-device; Top-Sided Stakeholder-Draft Guidance (TS-DG) which teaches “Intel Surveillance Recon” (ISR) best-practices; a Pocket-Sherpa; Technology as a Service (TaaS); L.G.B.T.Q.I.A+ honored and compassionate; the Pocket-Sherpa liberates subjugated patients, for use by Licensed Medical Intermediaries (LMIs), NPPs, conscripts, armchair clinics and DIY home-healthcare converts; deep-reserves of pre-positioned assets for family and emergency medicine; unique and unobvious; democratized Humanitarian-Aid on a Stick (HAS); declassified.
